SEQUENCE LISTING (4 sequences)

SEQ ID NO: 1 - DNA, 3416 bp, Homo sapiens
cggtacaggtaagtcggccgggcaggtaggggtgcccgaggagtagtcgctggagtccgcgcctccctgggactgcaatgtgccggtcttagctgctgcctgagaggatgtctggggtgtccgagcccctgagccgagtaaagttgggcacattacgccggcctgaaggccctgcagagcccatggtggtggtaccagtagatgtggaaaaggaggacgtgcgtatcctcaaggtctgcttctatagcaacagcttcaatcctgggaagaacttcaaactggtcaaatgcactgtccagacggagatccgggagatcatcacctccatcctgctgagcgggcggatcgggcccaacatccggttggctgagtgctatgggctgaggctgaagcacatgaagtccgatgagatccactggctgcacccacagatgacggtgggtgaggtgcaggacaagtatgagtgtctgcacgtggaagccgagtggaggtatgaccttcaaatccgctacttgccagaagacttcatggagagcctgaaggaggacaggaccacgctgctctatttttaccaacagctccggaacgactacatgcagcgctacgccagcaaggtcagcgagggcatggccctgcagctgggctgcctggagctcaggcggttcttcaaggatatgccccacaatgcacttgacaagaagtccaacttcgagctcctagaaaaggaagtggggctggacttgtttttcccaaagcagatgcaggagaacttaaagcccaaacagttccggaagatgatccagcagaccttccagcagtacgcctcgctcagggaggaggagtgcgtcatgaagttcttcaacactctcgccggcttcgccaacatcgaccaggagacctaccgctgtgaactcattcaaggatggaacattactgtggacctggtcattggccctaaagggatccgccagctgactagtcaggacgcaaagcccacctgcctggccgagttcaagcagatcaggtccatcaggtgcctcccgctggaggagggccaggcagtacttcagctgggcattgaaggtgccccccaggccttgtccatcaaaacctcatccctagcagaggctgagaacatggctgacctcatagacggctactgccggctgcagggtgagcaccaaggctctctcatcatccatcctaggaaagatggtgagaagcggaacagcctgccccagatccccatgctaaacctggaggcccggcggtcccacctctcagagagctgcagcatagagtcagacatctacgcagagattcccgacgaaaccctgcgaaggcccggaggtccacagtatggcattgcccgtgaagatgtggtcctgaatcgtattcttggggaaggcttttttggggaggtctatgaaggtgtctacacaaatcacaaaggggagaaaatcaatgtagctgtcaagacctgcaagaaagactgcactctggacaacaaggagaagttcatgagcgaggcagtgatcatgaagaacctcgaccacccgcacatcgtgaagctgatcggcatcattgaagaggagcccacctggatcatcatggaattgtatccctatggggagctgggccactacctggagcggaacaagaactccctgaaggtgctcaccctcgtgctgtactcactgcagatatgcaaagccatggcctacctggagagcatcaactgcgtgcacagggacattgctgtccggaacatcctggtggcctcccctgagtgtgtgaagctgggggactttggtctttcccggtacattgaggacgaggactattacaaagcctctgtgactcgtctccccatcaaatggatgtccccagagtccattaacttccgacgcttcacgacagccagtgacgtctggatgttcgccgtgtgcatgtgggagatcctgagctttgggaagcagcccttcttctggctggagaacaaggatgtcatcggggtgctggagaaaggagaccggctgcccaagcctgatctctgtccaccggtcctttataccctcatgacccgctgctgggactacgaccccagtgaccggccccgcttcaccgagctggtgtgcagcctcagtgacgtttatcagatggagaaggacattgccatggagcaagagaggaatgctcgctaccgaacccccaaaatcttggagcccacagccttccaggaacccccacccaagcccagccgacctaagtacagaccccctccgcaaaccaacctcctggctccaaagctgcagttccaggttcctgagggtctgtgtgccagctctcctacgctcaccagccctatggagtatccatctcccgttaactcactgcacaccccacctctccaccggcacaatgtcttcaaacgccacagcatgcgggaggaggacttcatccaacccagcagccgagaagaggcccagcagctgtgggaggctgaaaaggtcaaaatgcggcaaatcctggacaaacagcagaagcagatggtggaggactaccagtggctcaggcaggaggagaagtccctggaccccatggtttatatgaatgataagtccccattgacgccagagaaggaggtcggctacctggagttcacagggcccccacagaagcccccgaggctgggcgcacagtccatccagcccacagctaacctggaccggaccgatgacctggtgtacctcaatgtcatggagctggtgcgggccgtgctggagctcaagaatgagctctgtcagctgccccccgagggctacgtggtggtggtgaagaatgtggggctgaccctgcggaagctcatcgggagcgtggatgatctcctgccttccttgccgtcatcttcacggacagagatcgagggcacccagaaactgctcaacaaagacctggcagagctcatcaacaagatgcggctggcgcagcagaacgccgtgacctccctgagtgaggagtgcaagaggcagatgctgacggcttcacacaccctggctgtggacgccaagaacctgctcgacgctgtggaccaggccaaggttctggccaatctggcccacccacctgcagagtgacggagggtgggggccacctgcctgcgtcttccgcccctgcctgccatgtacctcccctgccttgctgttggtcatgtgggtcttccagggagaaggccaaggggagtcaccttcccttgccactttgcacgacgccctctccccacccctacccctggctgtactgctcaggctgcagctggacagaggggactctgggctatggacacagggtgacggtgacaaagatggctcagagggggactgctgctgcctggccactgctccctaagccagcct

SEQ ID NO: 2 - protein, 1009 aa, Homo sapiens
MSGVSEPLSRVKLGTLRRPEGPAEPMVVVPVDVEKEDVRILKVCFYSNSFNPGKNFKLVKCTVQTEIREIITSILLSGRIGPNIRLAECYGLRLKHMKSDEIHWLHPQMTVGEVQDKYECLHVEAEWRYDLQIRYLPEDFMESLKEDRTTLLYFYQQLRNDYMQRYASKVSEGMALQLGCLELRRFFKDMPHNALDKKSNFELLEKEVGLDLFFPKQMQENLKPKQFRKMIQQTFQQYASLREEECVMKFFNTLAGFANIDQETYRCELIQGWNITVDLVIGPKGIRQLTSQDAKPTCLAEFKQIRSIRCLPLEEGQAVLQLGIEGAPQALSIKTSSLAEAENMADLIDGYCRLQGEHQGSLIIHPRKDGEKRNSLPQIPMLNLEARRSHLSESCSIESDIYAEIPDETLRRPGGPQYGIAREDVVLNRILGEGFFGEVYEGVYTNHKGEKINVAVKTCKKDCTLDNKEKFMSEKVIMKNLDHPHIVKLIGIIEEEPTWIIMELYPYGELGHYLERNKNSLKVLTLVLYSLQICKAMAYLESINCVHRDIAVRNILVASPECVKLGDFGLSRYIEDEDYYKASVTRLPIKWMSPESINFRRFTTASDVWMFAVCMWEILSFGKQPFFWLENKDVIGVLEKGDRLPKPDLCPPVLYTLMTRCWDYDPSDRPRFTELVCSLSDVYQMEKDIAMEQERNARYRTPKILEPTAFQEPPPKPSRPKYRPPPQTNLLAPKLQFQVPEGLCASSPTLTSPMEYPSPVNSLHTPPLHRHNVFKRHSMREEDFIQPSSREEAQQLWEAEKVKMRQILDKQQKQMVEDYQWLRQEEKSLDPMVYMNDKSPLTPEKEVGYLEFTGPPQKPPRLGAQSIQPTANLDRTDDLVYLNVMELVRAVLELKNELCQLPPEGYVVVVKNVGLTLRKLIGSVDDLLPSLPSSSRTEIEGTQKLLNKDLAELINKMRLAQQNAVTSLSEECKRQMLTASHTLAVDAKNLLDAVDQAKVLANLAHPPAE

SEQ ID NO: 3 - DNA, 3416 bp, Recombinant Homo sapiens
cggtacaggtaagtcggccgggcaggtaggggtgcccgaggagtagtcgctggagtccgcgcctccctgggactgcaatgtgccggtcttagctgctgcctgagaggatgtctggggtgtccgagcccctgagccgagtaaagttgggcacattacgccggcctgaaggccctgcagagcccatggtggtggtaccagtagatgtggaaaaggaggacgtgcgtatcctcaaggtctgcttctatagcaacagcttcaatcctgggaagaacttcaaactggtcaaatgcactgtccagacggagatccgggagatcatcacctccatcctgctgagcgggcggatcgggcccaacatccggttggctgagtgctatgggctgaggctgaagcacatgaagtccgatgagatccactggctgcacccacagatgacggtgggtgaggtgcaggacaagtatgagtgtctgcacgtggaagccgagtggaggtatgaccttcaaatccgctacttgccagaagacttcatggagagcctgaaggaggacaggaccacgctgctctatttttaccaacagctccggaacgactacatgcagcgctacgccagcaaggtcagcgagggcatggccctgcagctgggctgcctggagctcaggcggttcttcaaggatatgccccacaatgcacttgacaagaagtccaacttcgagctcctagaaaaggaagtggggctggacttgtttttcccaaagcagatgcaggagaacttaaagcccaaacagttccggaagatgatccagcagaccttccagcagtacgcctcgctcagggaggaggagtgcgtcatgaagttcttcaacactctcgccggcttcgccaacatcgaccaggagacctaccgctgtgaactcattcaaggatggaacattactgtggacctggtcattggccctaaagggatccgccagctgactagtcaggacgcaaagcccacctgcctggccgagttcaagcagatcaggtccatcaggtgcctcccgctggaggagggccaggcagtacttcagctgggcattgaaggtgccccccaggccttgtccatcaaaacctcatccctagcagaggctgagaacatggctgacctcatagacggctactgccggctgcagggtgagcaccaaggctctctcatcatccatcctaggaaagatggtgagaagcggaacagcctgccccagatccccatgctaaacctggaggcccggcggtcccacctctcagagagctgcagcatagagtcagacatctacgcagagattcccgacgaaaccctgcgaaggcccggaggtccacagtatggcattgcccgtgaagatgtggtcctgaatcgtattcttggggaaggcttttttggggaggtctatgaaggtgtctacacaaatcacaaaggggagaaaatcaatgtagctgtcgcgacctgcaagaaagactgcactctggacaacaaggagaagttcatgagcgaggcagtgatcatgaagaacctcgaccacccgcacatcgtgaagctgatcggcatcattgaagaggagcccacctggatcatcatggaattgtatccctatggggagctgggccactacctggagcggaacaagaactccctgaaggtgctcaccctcgtgctgtactcactgcagatatgcaaagccatggcctacctggagagcatcaactgcgtgcacagggacattgctgtccggaacatcctggtggcctcccctgagtgtgtgaagctgggggactttggtctttcccggtacattgaggacgaggactattacaaagcctctgtgactcgtctccccatcaaatggatgtccccagagtccattaacttccgacgcttcacgacagccagtgacgtctggatgttcgccgtgtgcatgtgggagatcctgagctttgggaagcagcccttcttctggctggagaacaaggatgtcatcggggtgctggagaaaggagaccggctgcccaagcctgatctctgtccaccggtcctttataccctcatgacccgctgctgggactacgaccccagtgaccggccccgcttcaccgagctggtgtgcagcctcagtgacgtttatcagatggagaaggacattgccatggagcaagagaggaatgctcgctaccgaacccccaaaatcttggagcccacagccttccaggaacccccacccaagcccagccgacctaagtacagaccccctccgcaaaccaacctcctggctccaaagctgcagttccaggttcctgagggtctgtgtgccagctctcctacgctcaccagccctatggagtatccatctcccgttaactcactgcacaccccacctctccaccggcacaatgtcttcaaacgccacagcatgcgggaggaggacttcatccaacccagcagccgagaagaggcccagcagctgtgggaggctgaaaaggtcaaaatgcggcaaatcctggacaaacagcagaagcagatggtggaggactaccagtggctcaggcaggaggagaagtccctggaccccatggtttatatgaatgataagtccccattgacgccagagaaggaggtcggctacctggagttcacagggcccccacagaagcccccgaggctgggcgcacagtccatccagcccacagctaacctggaccggaccgatgacctggtgtacctcaatgtcatggagctggtgcgggccgtgctggagctcaagaatgagctctgtcagctgccccccgagggctacgtggtggtggtgaagaatgtggggctgaccctgcggaagctcatcgggagcgtggatgatctcctgccttccttgccgtcatcttcacggacagagatcgagggcacccagaaactgctcaacaaagacctggcagagctcatcaacaagatgcggctggcgcagcagaacgccgtgacctccctgagtgaggagtgcaagaggcagatgctgacggcttcacacaccctggctgtggacgccaagaacctgctcgacgctgtggaccaggccaaggttctggccaatctggcccacccacctgcagagtgacggagggtgggggccacctgcctgcgtcttccgcccctgcctgccatgtacctcccctgccttgctgttggtcatgtgggtcttccagggagaaggccaaggggagtcaccttcccttgccactttgcacgacgccctctccccacccctacccctggctgtactgctcaggctgcagctggacagaggggactctgggctatggacacagggtgacggtgacaaagatggctcagagggggactgctgctgcctggccactgctccctaagccagcct

SEQ ID NO: 4 - protein, 1009 aa, Recombinant Homo sapiens
MSGVSEPLSRVKLGTLRRPEGPAEPMVVVPVDVEKEDVRILKVCFYSNSFNPGKNFKLVKCTVQTEIREIITSILLSGRIGPNIRLAECYGLRLKHMKSDEIHWLHPQMTVGEVQDKYECLHVEAEWRYDLQIRYLPEDFMESLKEDRTTLLYFYQQLRNDYMQRYASKVSEGMALQLGCLELRRFFKDMPHNALDKKSNFELLEKEVGLDLFFPKQMQENLKPKQFRKMIQQTFQQYASLREEECVMKFFNTLAGFANIDQETYRCELIQGWNITVDLVIGPKGIRQLTSQDAKPTCLAEFKQIRSIRCLPLEEGQAVLQLGIEGAPQALSIKTSSLAEAENMADLIDGYCRLQGEHQGSLIIHPRKDGEKRNSLPQIPMLNLEARRSHLSESCSIESDIYAEIPDETLRRPGGPQYGIAREDVVLNRILGEGFFGEVYEGVYTNHKGEKINVAVKTCKKDCTLDNKEKFMSEAVIMKNLDHPHIVKLIGIIEEEPTWIIMELYPYGELGHYLERNKNSLKVLTLVLYSLQICKAMAYLESINCVHRDIAVRNILVASPECVKLGDFGLSRYIEDEDYYKASVTRLPIKWMSPESINFRRFTTASDVWMFAVCMWEILSFGKQPFFWLENKDVIGVLEKGDRLPKPDLCPPVLYTLMTRCWDYDPSDRPRFTELVCSLSDVYQMEKDIAMEQERNARYRTPKILEPTAFQEPPPKPSRPKYRPPPQTNLLAPKLQFQVPEGLCASSPTLTSPMEYPSPVNSLHTPPLHRHNVFKRHSMREEDFIQPSSREEAQQLWEAEKVKMRQILDKQQKQMVEDYQWLRQEEKSLDPMVYMNDKSPLTPEKEVGYLEFTGPPQKPPRLGAQSIQPTANLDRTDDLVYLNVMELVRAVLELKNELCQLPPEGYVVVVKNVGLTLRKLIGSVDDLLPSLPSSSRTEIEGTQKLLNKDLAELINKMRLAQQNAVTSLSEECKRQMLTASHTLAVDAKNLLDAVDQAKVLANLAHPPAE

(19) United States
(12) Patent Application Publication
(10) Pub. No.: US 2009/0227656 A1
(43) Pub. Date: Sep. 10, 2009

(54) METHODS FOR USING MODULATORS OF PROLINE-RICH TYROSINE KINASE 2

(75) Inventors: Irene Griswold-Prenner, San Francisco, CA (US); Kyle Powell, Oakland, CA (US)

Correspondence Address: Townsend and Townsend and Crew LLP, Two Embarcadero Center, Eighth Floor, San Francisco, CA, 94111, US

(73) Assignee: Elan Pharmaceuticals, Inc., South San Francisco, CA (US)

(21) Appl. No.: 12/403,342

(22) Filed: Mar. 12, 2009

Related U.S. Application Data
(63) Continuation of application No. 10/860,066, filed on Jun. 4, 2004, now abandoned.
(60) Provisional application No. 60/476,178, filed on Jun. 6, 2003.

(51) Int. Cl. A61K 31/7088; A61P 25/28; A61P 25/16

(52) U.S. Cl. 514/44 A

(57) ABSTRACT

The present invention relates to methods for preventing cell death in a subject and their application in the treatment of neurodegenerative diseases and conditions, such as Alzheimer's disease, stroke, Parkinson's disease etc. A method for preventing cell death comprises reducing or inhibiting Pyk2 activity.

### RELATED APPLICATIONS

This application claims the benefit of U.S. Provisional Application No. 60/476,178, filed Jun. 6, 2003, which is herein incorporated by reference.

### FIELD OF THE INVENTION

The present invention is related generally to methods for preventing neuronal cell death by the administration of a modulator of proline-rich tyrosine kinase 2 (Pyk2). Modulators of Pyk2 may be used for the treatment of a variety of conditions associated with neurodegeneration.

### BACKGROUND OF THE INVENTION

Neuronal death, whether occurring naturally during development or resulting from pathological conditions, can result from a variety of causes. It was generally believed that different causes of neuronal death must involve different mechanisms. For example, natural and pathological neuronal death have been attributed, respectively, to apoptosis and necrosis, which are morphologically distinct. Differences in the time course of cell death have also been inferred to indicate that different mechanisms are involved. Despite these apparent differences in the process of neuronal death, it is has been suggested that multiple initiating causes may converge at some point in the cascade of events leading to neuronal injury and death (Mattson et al., 1993, Ann. N.Y. Acad. Sci. 679:1-21).

For example, a number of adult-onset diseases, and in particular neurodegenerative diseases, are associated with abnormal precipitation and/or aggregation of proteins. Examples include Alzheimer's disease, which exhibits neurofibrillary tangles (NFT), senile plaques, and cerebrovascular deposits of amyloid-beta; Parkinson's disease, which exhibits Lewy bodies; prion disorders, which exhibit plaques comprising prion proteins; Huntington's disease, which exhibits huntington precipitates; dominantly inherited spinocerebellar ataxias, which exhibit corresponding ataxin protein precipitates; multiple system atrophy, which exhibits alpha-synuclein deposits; progressive supranuclear palsy, which exhibits tau precipitates; and familial amyotrophic lateral sclerosis, which exhibits superoxide dimutase 1 (SOD1) precipitates. See, e.g., Johnson, 2000, J. Anat. 196:609-616. As these varied diseases display many pathological mechanisms in common, it is possible that they share pathways that lead to aberrant protein aggregation and/or precipitation in these disorders (Hardy and Gwinn-Hardy, 1998, Science 282:1075-1079).

Regulation of intracellular calcium is also thought to play an important role in neuronal structure and function. Neurons possess elaborate systems for regulating intracellular calcium levels and responding to changes. Ligand-gated Ca²⁺ channels such as the excitatory amino acid receptors, which include the N-methyl-d-aspartate receptor (NMDA) and the alpha-amino-3-hydroxy-5-methyl-4-isoxazolepropionate (AMPA) receptor, and voltage-dependent calcium channels provide the major sites of Ca²⁺ influx into neurons of brain regions, such as the hippocampus, that are vulnerable in stroke and AD. Ca²⁺ is normally removed from the cytoplasm by the plasma membrane Na⁺/Ca²⁺ exchanger. Loss of regulation of intracellular calcium levels that results in an elevated Ca²⁺ concentration can cause structural damage to neurons and initiate the process of cell death. Data indicate that excessive elevations of intracellular Ca²⁺ levels may be involved in the neuronal degeneration that is observed in traumatic neuronal injury, stroke, AD, and Huntington's disease. Some studies suggest that activating potassium (K⁺) channels may prevent some of the damage caused by elevated intracellular Ca²⁺ levels. Other methods for reducing Ca²⁺-induced neurotoxicity include administration of NMDA antagonists, AMPA antagonists, chelators of intracellular Ca²⁺, free radical scavengers, sodium channel antagonists, glutamate release inhibitors, growth factors, and hypothermia.

A brief overview of exemplary neurodegenerative disorders is provided below.

### Alzheimer's Disease

Alzheimer's disease (AD) is the leading cause of senile dementia, affecting more than 4 million people in the United States alone. AD is characterized by a progressive loss of memory, cognitive impairment, loss of social appropriateness, and decline in capacity to communicate. The major pathological lesions described in AD patients included senile plaques (SP) and neurofibrillary tangles (NFT). However, these lesions were initially considered a consequence rather than a cause of the disease. Scientists have since found that the major component of the core of the SP is a novel 42-residue peptide called amyloid β-peptide 42 (Aβ42), which is cleaved from a larger precursor protein, APP (Haass et al., 1991, J. Neurosci. 11:3783-3793; Haass et al., 1993, J. Biol. Chem. 268:3021-3024; Turner et al., 1996, J. Biol. Chem. 142:1274-1280). Other forms of β-amyloid protein (Aβ) having 39-42 residues have also been found, all originating from APP and contributing to senile plaques. It is now believed that Aβ is a major contributor to the pathogenesis in AD.

Three important enzymes collectively known as “secretases” participate in APP processing and play critical roles in the generation of Aβ. β-secretase cleaves APP at the amino terminus, producing a large secreted derivative, sAPPβ, and an Aβ-bearing membrane-associated C-terminal derivative, CTFβ, which is subsequently cleaved by γ-secretase to release Aβ. α-secretase cleaves APP within Aβ to produce a secreted derivative, sAPPα, and a membrane-associated derivative, CTFα. Initially, methods for treating AD focused on increasing α-secretase activity because it prevents Aβ accumulation. α-secretase activity may be stimulated by phorbol esters, growth factors, cytokines, and neurotransmitters. Many of these activate kinases, including tyrosine kinases, such as protein kinase C and mitogen-activated protein kinase (MAPK), which in turn increase α-secretase activity. This approach has not been successful, and often resulted in increases in sAPPα without reduction in Aβ accumulation. Sometimes, even increases in Aβ accumulation were observed. Thus, there remains a need for a method of treating AD.

### Stroke

Stroke is an ischemic/excitotoxic condition arising from circulatory impairment and often involving irreversible brain damage. The outcome of a stroke varies from minimal impairment to rapid onset of coma followed by death. Stroke is the third leading cause of death in adults in the United States.

Severe, acute ischemia in nerve tissue triggers cellular changes, such as calcium influx and protease activation, that can swiftly cause irreversible damage. The NMDA receptor, which is activated by glutamate, is probably the major site of calcium entry in ischemic/excitotoxic conditions, although calcium influx through non-NMDA receptors, such as voltage-dependent calcium channels, may also play a role in stroke.

Early and aggressive treatment of stroke is crucial in limiting damage to brain tissue. Current treatments include intravenous administration of tissue plasminogen activator (TPA) within the first three hours of the onset of the stroke, to dissolve any obstructing thrombus. Intravenous administration of thrombolytic agents other than TPA has proven less effective and more likely to cause hemorrhage. Prourokinase, hypothermia, and heparin plus magnesium have also proven somewhat beneficial in selected cases. Long-term outcome, however, may depend on the aggressiveness and persistence of physical therapy and rehabilitation.

### Parkinson's Disease

Parkinson's disease (PD) is one of the most common neurodegenerative diseases, with a prevalence of about 1% in people 65 years of age, but increasing to 4%-5% by age 85. The major clinical symptoms of Parkinson's disease include bradykinesia, resting tremor, rigidity, and postural instability. The underlying pathology of the disease is a profound reduction in striatal dopamine content caused by the death of dopaminergic neurons in the substantia nigra (SN) pars compacta (Forno, 1996, Neuropathol. Exp. Neurol. 55:259-272). Disease symptoms appear after degeneration of more than 70-80% of the dopaminergic neurons in the substantia nigra.

Broadly speaking, the disease falls into two categories: late onset and early onset PD. Late onset PD is observed predominantly in patients over the age of 55 and is believed to be for the most part the product of environmental factors. Late onset PD is characterized by dopaminergic neuron death at a faster rate, and to a more severe degree, than in healthy individuals. Early onset Parkinson's disease is less frequent, with symptoms developing between the ages of 35 and 60.

PD's distinct pathological lesions of the SN include eosinophilic filamentous inclusions known as Lewy bodies (LBs) and dystrophic neuritis, termed Lewy neurites (LNs). Id. The major components of LBs are α-synuclein and ubiquitin. Over the last few years, two mutations in α-synuclein have been found to be associated with familial cases of PD, highlighting the importance of α-synuclein and LBs in the pathogenesis of PD (Polymeropoulos et al., 1997, Science 276:2045-2047).

At present, the most common therapies for PD involve attempts to increase the dopamine content in patients via the administration of L-DOPA (as a precursor of dopamine), dopamine agonists, or monoamino oxidase B inhibitors (blocking the degradation of dopamine). These treatments alleviate some of the disease symptoms, but, because dopamineric neurons continue to die, they do not prevent the progression of the disease. At present, there are no prophylactic therapies available to stop the progression of this neurodegenerative disease.

In view of the above points, there is clearly a need for methods of treating and/or preventing the cell death associated with neurodegenerative disorders and conditions. Such a method would be effective in the treatment, prevention, and/or reversal of some of the conditions exhibited in subjects suffering from such disorders.

### SUMMARY OF THE INVENTION

The present invention relates to methods for preventing cell death. In general, the methods of the invention may be practiced in vivo or in vitro. When practiced in vivo, the methods of the invention provide effective treatment for the condition suffered by a subject. When practiced in vitro, the methods of the invention provide a better understanding of the particular disease in question (Alzheimer's disease, stroke, Parkinson's disease, etc.), which will allow improved diagnosis and treatment of these diseases.

In one embodiment of the invention, a method for preventing cell death comprises modulating the activity of Pyk2. The activity of Pyk2 may be modulated by introducing a Pyk2 inhibitor or a Pyk2 activator into a cell. A Pyk2 inhibitor may include, for example, phosphatases, dominant-negative forms of Pyk2, chemicals, or other naturally-occurring, recombinant, or synthetic nucleic acid, protein, polypeptide, or polysaccharide agent that reduces or inhibits Pyk2 activity. A Pyk2 activator may include, for example, phosphatase inhibitors, neurotransmitters, calcium ionophores, chemicals, or other naturally-occurring, recombinant, or synthetic nucleic acid, protein, polypeptide, or polysaccharide agent that enhances or activates Pyk2 activity.

Thus, an embodiment of the invention includes a method for preventing cell death comprising introducing a Pyk2 modulator into a cell, wherein the Pyk2 modulator is a Pyk2 inhibitor or Pyk2 activator.

In another embodiment of the invention, the method for preventing cell death comprises introducing a vector comprising a nucleic acid sequence encoding a Pyk2 modulator into a cell by methods known in the art. This nucleic acid sequence may be operably linked to one or more transcriptional regulatory sequences, such as promoters, enhancers, activators, terminators, polyadenylation signals, and other regulatory sequences known to the skilled artisan.

In an embodiment of the invention, the nucleic acid sequence encodes a Pyk2 inhibitor. In one embodiment, a nucleic acid encoding a Pyk2 inhibitor may comprise antisense oligonucleotides directed to Pyk2 sequences. In a further embodiment, a nucleic acid encoding a Pyk2 inhibitor may comprise RNAi (interfering RNA). In another embodiment, a nucleic acid encoding a Pyk2 inhibitor may encode a dominant-negative Pyk2. In yet another embodiment of the invention, a nucleic acid encoding a Pyk2 inhibitor may encode a protein, peptide, or polypeptide that will bind to and reduce or inhibit the activity of Pyk2.

Another embodiment of the invention relates to compositions suitable for preventing cell death.

In an embodiment of the invention, the composition comprises a Pyk2 modulator. The Pyk2 modulator may be a Pyk2 inhibitor or activator. Compositions optionally comprise appropriate physiologically and/or pharmaceutically acceptable additives, lubricants, diluents, buffers, moistening agents, preservative agents, flavorings, adjuvants, carriers, stabilizers, suspending agents, emulsifying agents, propellants, and/or other vehicles.

In another embodiment of the invention, a method for preventing cell death in a patient comprises administering a Pyk2 modulator into the patient, wherein the Pyk2 modulator may be a Pyk2 inhibitor or activator. In an embodiment of the invention, a patient may have Alzheimer's disease, have suffered from a stroke, Parkinson's disease, or any other disease or condition associated with neurodegeneration or neuronal cell death.

Additional embodiments and advantages of the invention will be set forth in part in the detailed description that follows, and in part will be apparent upon considering the application as a whole. Additionally, further embodiments and advantages of the invention may also be learned by practicing the invention.

### BRIEF DESCRIPTION OF THE DRAWINGS

FIGS. 1A-D show the DNA sequence of human Pyk2 (SEQ ID NO: 1)

FIGS. 2A-I show the amino acid sequence of human Pyk2 (SEQ ID N0: 2)

FIGS. 3A-D show the DNA sequence of dominant-negative human Pyk2 (SEQ ID NO: 3)

FIGS. 4A-I show the amino acid sequence of dominant-negative human Pyk2 (SEQ ID NO: 4)

FIG. 5 shows the percent inhibition of Aβ-induced neurotoxicity by the dominant-negative human Pyk2 (DN Pyk2) and dominant-negative human JNK3 (DN JNK) compared with the control, green fluorescent protein (GFP). Figure shows the results of three different experiments.

### DETAILED DESCRIPTION OF THE INVENTION

The embodiments described and the terminology used herein are for the purpose of describing exemplary embodiments only, and are not intended to be limiting. The scope of the present invention is intended to encompass additional embodiments not specifically described herein, but that would be apparent to one skilled in the art upon reading the present disclosure and practicing the invention.

Other than in the operating examples, or where otherwise indicated, all numbers expressing quantities of ingredients, reaction conditions, and so forth used in this application are to be understood as being modified in all instances by the term “about.” Accordingly, unless the contrary is indicated, the numerical parameters set forth in this application are approximations that may vary depending upon the desired properties sought to be obtained by the present invention. At the very least, and not as an attempt to limit the application of the doctrine of equivalents to the scope of the claims, each numerical parameter should be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements.

Unless defined otherwise, all technical and scientific terms used herein have the same meaning as commonly understood by one of ordinary skill in the art to which this invention belongs. Although any methods and materials similar or equivalent to those described herein can also be used in the practice of the present invention, exemplary methods and materials are described for illustrative purposes.

All publications mentioned in this application are incorporated by reference to disclose and describe the methods and/or materials in connection with which the publications are cited. Additionally, the publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

It must be noted that as used herein and in the appended claims, the singular forms “a”, “an”, and “the” include plural referents unless the context clearly dictates otherwise. Thus, for example, reference to “an antibody” includes a plurality of antibodies and reference to “a vector” includes reference to one or more vectors and equivalents thereof known to those skilled in the art.

Methods, techniques, and/or protocols (collectively “methods”) that can be used in the practice of the invention are not limited to the particular examples of these procedures cited throughout the specification but embrace any procedure known in the art for the same purpose. For example, with respect to methods for the delivery of nucleic acids to the brain, the present invention is not limited to the protocols cited herein, but includes any method available in the art to the skilled artisan to deliver nucleic acids to brain cells.

Furthermore, although some methods may be described in a particular context in the specification, their use in the instant invention is not limited to that context.

### DEFINITIONS

The term “Pyk2 modulator” refers to any chemical or naturally-occurring, recombinant, or synthetic nucleic acid, protein, polypeptide, or polysaccharide that enhances, activates, reduces, or inhibits any Pyk2 activity or the expression of Pyk2. “Pvk2 activity” may include, but is not limited to, phosphorylation of potassium ion channels, suppression of potassium ion channels, activation of MAP kinase or the MAP kinase signaling pathway, or transcriptional regulation.

The terms “inhibitor of Pyk2” and “Pyk2 inhibitor” refer to any chemical or naturally-occurring, recombinant, or synthetic nucleic acid, protein, polypeptide, or polysaccharide that reduces or inhibits any Pyk2 activity, reduces or inhibits tyrosine phosphorylation of Pyk2, or reduces or inhibits transcription and/or translation of Pyk2. Examples of Pyk2 inhibitors include, but are not limited to, phosphatases, dominant-negative Pyk2, and antisense oligonucleotides directed to a nucleic acid encoding Pyk2.

The terms “activator of Pyk2” and “Pvk2 activator” refer to any chemical or naturally-occurring, recombinant, or synthetic nucleic acid, protein, polypeptide, or polysaccharide that activates or enhances any Pyk2 activity, activates or enhances tyrosine phosphorylation of Pyk2, or activates or enhances transcription and/or translation of Pyk2. Examples of Pyk2 activators include, but are not limited to, phosphatase inhibitors, neurotransmitters, and compounds such as calcium ionophores that promote calcium influx.

The term “dominant negative Pyk2” refers to a form of Pyk2 that interferes with the functioning of the normally active Pyk2. The functioning of the normally active Pyk2 may be at the transcriptional, translational, or protein level. An example of a dominant negative Pyk2 is presented in Lev et al., 1995, Nature 376:737-745 and in Tokiwa et al., 1996, Science 273:792-794.

The terms “nucleic acid” and “polynucleotide” refer to a polymer of at least two nucleotides. The term nucleic acid includes deoxyribonucleic acid (DNA) and ribonucleic acid (RNA). RNA includes, but is not limited to, tRNA (transfer RNA), snRNA (small nuclear RNA), rRNA (ribosomal RNA), mRNA (messenger RNA), anti-sense RNA, and ribozymes. Similarly, DNA includes, but is not limited to, plasmid DNA, viral DNA, linear DNA, chromosomal DNA, or derivatives of these groups. In addition, these forms of DNA and RNA may be single, double, triple, or quadruple stranded. The term nucleic acid also includes PNAs (peptide nucleic acids), phosphorothioates, and other variants of the phosphate backbone of native nucleic acids. In some cases, when a nucleic acid encodes a polypeptide to be introduced into a cell, it may be advantageous, although not necessary, that the nucleic acid be in the form of a double-stranded cDNA lacking intron segments.

The terms “peptide”, “polypeptide”, and “protein” refer to chains comprising two or more amino acids covalently joined by peptide bonds. Generally, “peptide” refers to a chain comprising a small number of amino acids, while “polypeptide” refers to a chain comprising many amino acids, and “protein” refers to a macromolecule comprising one or more polypeptide chains. Although the terms “polypeptide” and “protein” are sometimes used interchangeably, molecules referred to as “polypeptides” generally have molecular weights below 10,000, and “proteins” generally have molecular weights above 10,000. Because there is no exact size range for determining whether an amino acid chain is a “peptide”, “polypeptide”, or “protein”, “peptide” and “polypeptide” or “polypeptide” and “protein” are sometimes used interchangeably.

The term “antisense oligonucleotide” refers to a nucleic acid that modulates the function of a nucleic acid molecule encoding a protein, ultimately modulating the amount of protein produced. Modulation of the amount of protein produced is accomplished by providing antisense oligonucleotides that specifically hybridize with one or more nucleic acids encoding the protein. As used herein, the terms “target nucleic acid” and “nucleic acid encoding a protein” encompass DNA encoding the protein, RNA (including pre-mRNA and mRNA) transcribed from such DNA, and also cDNA derived from such RNA. The specific hybridization of an antisense oligonucleotide with its target nucleic acid interferes with the normal function of the nucleic acid. This modulation of function of a target nucleic acid by compounds that specifically hybridize to it is generally referred to as “antisense.” The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of the protein. In the context of the present invention, “modulation of the expression of the protein” means either an increase (stimulation) or a decrease (inhibition) in the expression of a protein.

The term “RNAi” or “interfering RNA” refers to RNA that is partially or fully double-stranded similar to a portion of the target nucleic acid. When RNAi enters a cell, it triggers a cellular process that causes the degradation of not only the invading dsRNA molecule, but also single-stranded (ssRNAs) RNAs of identical sequences, including endogenous mRNAs. Thus, the overall effect of RNAi on target nucleic acid function is a decrease or inhibition of the expression of the target protein.

A nucleic acid may also be constructed to express a whole or partial protein. Once a polynucleotide has been delivered to a cell, the nucleic acid can remain separate from the endogenous genetic material. Alternatively, the nucleic acid could recombine with (become a part of) the endogenous genetic material. For example, DNA can be inserted into chromosomal or genomic DNA by either homologous or nonhomologous recombination. Homologous recombination is described, for example, in U.S. Pat. Nos. 5,282,071, and 5,578,461. Nonhomologous recombination is described, for example, in PCT Application No. WO 00/49162.

Optionally, depending on their particular function, nucleic acid molecules may be operably linked to one or more transcriptional regulatory sequences.

One embodiment of the term vector refers to a polynucleotide having a nucleotide sequence that can assimilate other nucleic acid sequences, and propagate those sequences in an appropriate host. A vector may be capable of self-replication or may simply serve as a carrier for a given nucleic acid sequence. Vectors may originate from viruses, plasmids, or the cells of a higher organism, and often contain DNA sequences from several sources. Examples of vectors include plasmids, cosmids, and yeast artificial chromosomes. Examples of viral vectors include adenovirus vectors, adenoassociated viral vectors (AAV), retrovirus vectors, herpes virus vectors, vaccinia virus vectors, and RNA virus vectors. Non-viral vectors include expression plasmid vectors typically used in mammalian cells, and are exemplified by pBK-CMV, pCAGGS, pcDNA3.1, pZeoSV, and the like. See, e.g., European Patent Application Nos. 1 122 312 and 1 132 098.

Another embodiment of the term vector refers to a non-nucleic acid carrier used to introduce a nucleic acid sequence into a cell. This type of vector may form complexes with the nucleic acid sequence, for example, an antisense oligonucleotide complexed to protein, an antisense oligonucleotide complexed with lipid-based nucleic acid transduction systems, or other non-viral carrier systems. Suitable lipids and related analogs are described in U.S. Pat. Nos. 5,208,036, 5,264,618, 5,279,833, and 5,283,185. Vectors can also be particulate carriers, examples of which include polymethyl methacrylate polymers and polylactides and poly(lactide-co-glycolides). See, e.g., McGee et al., 1997, J. Microencapsul. 14:197-210.

Several non-viral methods for the transfer of polynucleotides into cultured mammalian cells are also contemplated by the present invention, and include, without being limited to, calcium phosphate precipitation; DEAE-dextran; electroporation; direct microinjection; DNA-loaded liposomes; and receptor-mediated transfection. These techniques are disclosed in, e.g., PCT patent application WO 01/42451. Some of these techniques may be successfully adapted for in vivo or ex vivo use.

Vectors are normally used to mediate the expression of a molecule of interest in a cell, in which case they are called expression vectors. An “expression vector” is a vector in which the nucleic acid sequence encoding a molecule of interest is operably linked to suitable transcriptional regulatory sequences capable of effecting the expression of the molecule of interest in a cell.

A nucleic acid is operably linked when it is placed into a functional relationship with another nucleic acid sequence. For example, a promoter or enhancer is operably linked to a coding sequence if it affects the transcription of the sequence.

A transcriptional regulatory sequence is a nucleic acid sequence that regulates the expression of a nucleic acid sequence to which it is operably linked. A regulatory sequence may include elements that are naturally responsible for expressing a particular nucleic acid or may include sequences of a different origin. In general, regulatory sequences can be sequences of eukaryotic or viral genes or derived sequences that stimulate or repress transcription of a gene in a specific or non-specific manner and in an inducible or non-inducible manner. Transcriptional regulatory sequences include, e.g., promoters, RNA splice sites, enhancers, and transcriptional termination sequences. As the skilled artisan will appreciate, regulatory sequences may be constitutive or inducible depending on the particular nature of the sequence and the role of the nucleic acid sequence operably linked to the regulatory sequence.

A promoter is a transcriptional regulatory sequence that directs RNA polymerase to bind to DNA and initiate RNA synthesis. A strong promoter is one that causes mRNAs to be initiated at high frequency. Examples of promoters that can be used in the construction of vectors for the delivery of nucleic acid sequences to neuronal tissue include the promoter/enhancer from the human cytomegalovirus (hCMV) (WO 98/46273), and the simian virus 40 (SV40) promoter (Harada et al., 2000, Cancer Gene Ther. 7:799-805). Other promoters that cause expression in the brain include the platelet-derived growth factor (PDGF) promoter (WO 02/26936); prion promoter (Hsiao et al., 1996, Science 274:99-102); and the neuron-specific enolase promoter (Xu et al., 2001, Gene Ther. 8:1323-32). Other promoters known in the art may also be used.

An enhancer is a transcriptional regulatory sequence that increases transcription by a promoter. Enhancers can effectively increase transcription when located either 5′ or 3′ to the transcription unit. They are also effective if located within an intron or within the coding sequence itself. Typically, viral enhancers may be used, including SV40 enhancers, cytomegalovirus enhancers, polyoma enhancers, and adenovirus enhancers. See, e.g., WO 98/55616 and U.S. Pat. Nos. 6,248,555 and 6,323,030. Enhancer sequences from mammalian systems are also commonly used, such as the mouse immunoglobulin heavy chain enhancer. See, e.g., WO 92/04440. Other enhancers known in the art may also be used.

A transcriptional terminator sequence refers either to a sequence that provides a signal that terminates transcription by the polymerase that recognizes the selected promoter or to a signal sequence for polyadenylation. These terminator sequences may be isolated from bacteria, fungi, viruses, animals and/or plants. In the case of terminators from prokaryotic cells, the terminator generally includes a polyadenylation signal, which facilitates the addition of polyadenylate sequences to the 3′ end of a primary transcript. Examples of terminators include those from the cytomegalovirus and SV40 systems, as well as the bovine growth hormone (BGH) polyadenylation sequence. See, e.g., WO 98/55616, WO 02/16594 and U.S. Pat. Nos. 6,248,555 and 6,323,030.

Mammalian expression vector systems may include a selectable marker gene. Examples of suitable markers include the dihydrofolate reductase gene (DHFR), the thymidine kinase gene (TK), fluorescent green protein, β-galactosidase, or prokaryotic genes conferring drug resistance. The first two marker genes are normally used with mutant cell lines that lack the ability to grow without the addition of thymidine to the growth medium. Transformed cells can then be identified by their ability to grow on non-supplemented media. Examples of prokaryotic drug resistance genes useful as markers include genes conferring resistance to G418, mycophenolic acid, and hygromycin. See, e.g., U.S. Pat. No. 6,165,793

A therapeutically beneficial effect of a modification of an original host cell is obtained when the modified host cell shows a measurable improvement in a desired characteristic (e.g., prevention of cell death) with respect to the original host control. A therapeutically beneficial effect may come about, for example, as a result of modulating Pyk2 activity.

In the context of the invention, a cell in need of a method for preventing cell death is a cell in which undesired neuronal cell death occurs. For example, such a cell may include an ischemic cell. Other examples may include cells around which neurofibrillary tangles (NFT) or senile plaques (SP) accumulate, for instance in patients with Alzheimer's disease, etc. Similarly, a subject in need of a method for preventing cell death is a subject in which undesired neuronal cell death occurs.

The terms effective dose, effective amount, therapeutically effective amount, and the like mean an amount of an agent sufficient to provide the desired physiological, pharmacological, and/or cognitive change. This will vary depending on the patient, the disease, and the treatment. The amount may either be a dose for the treatment of a subject believed to have a particular disorder, in which case it should sufficiently alleviate or ameliorate the symptoms of the disorder or condition, or be a prophylactic dose, in which case it should be sufficient to prevent, partially or completely, the appearance of symptoms in the subject.

The terms treatment, treating and the like are used herein to mean obtaining a desired pharmacologic and/or physiologic effect. The effect may be prophylactic in terms of completely or partially preventing a disease or a symptom thereof and/or may be therapeutic in terms of a partial or complete cure for a disease and/or an adverse effect attributable to the disease. Treatment as used herein covers any treatment of a disease in a patient and includes:

(a) preventing the disease from occurring in a subject that may be predisposed to the disease but not yet diagnosed as having it;
(b) altering the progression of the disease, i.e., slowing or arresting its development; or
(c) reducing the severity of the disease, i.e., causing regression of the disease or alleviating one or more symptoms of the disease.

A patient is hereby defined as any person or non-human animal in need of treatment with a specific chemical, nucleic acid, protein, polypeptide, peptide, or polysaccharide or to any subject for whom treatment may be beneficial, including humans and non-human animals. Such non-human animals to be treated include all domesticated and feral vertebrates. One of skill in the art will, of course, recognize that the choice of chemical, nucleic acid, protein, polypeptide, peptide, or polysaccharide will depend on the disease or condition to be treated in a particular system.

### GENERAL ASPECTS OF THE INVENTION

The present invention relates to methods for preventing cell death. For example, preventing neuronal cell death is contemplated within the present invention, including preventing neuronal cell death in a patient.

In general, the methods of the invention may be practiced in vivo or in vitro. When practiced in vivo, the methods of the invention will provide effective treatment for the condition suffered by the subject. When practiced in vitro, the methods of the invention will provide a better understanding of the particular disease or condition in question (Alzheimer's disease, stroke, etc.) that will allow improvement of the diagnosis and treatment of these diseases.

The present invention is based on the finding that inhibitors of Pyk2 are capable of preventing or inhibiting neuronal cell death. “Pyk2” is a non-receptor protein tyrosine kinase belonging to the same family of protein tyrosine kinases as the focal adhesion kinase (FAK). FAK is a cytoplasmic protein kinase localized to focal adhesions and contains a focal adhesion targeting (FAT) domain in its C-terminus. Pyk2 also contains a FAT domain. FAK and Pyk2 show 52% sequence similarity overall, and the FAT domains show 62% sequence similarity. Both Pyk2 and FAK are abundantly expressed in the adult brain, with distinctive but partially overlapping distributions. Pyk2 is expressed predominantly in the brain in discrete populations of neurons, with the highest levels observed in the hippocampus, dentate gyrus and olfactory bulb (Lev et al., 1995, Nature 376:737-745). Moreover, Pyk2 is expressed at low levels in the embryonic brain and becomes very abundant in the adult brain (Menegon et al., 1999, Eur. J. Neurosci. 11:3777-3788; Girault et al., 1999, Trends in Neurosciences 22:257-263). In contrast with Pyk2, the highest levels of FAK are observed at the end of the embryonic life (Girault et al., 1999, Trends in Neurosciences 22:257-263). In adult rats, FAK is found in high levels in the cerebral cortex and in the hippocampus (Worley and Holt, 1996, NeuroReport 7:1133-1137).

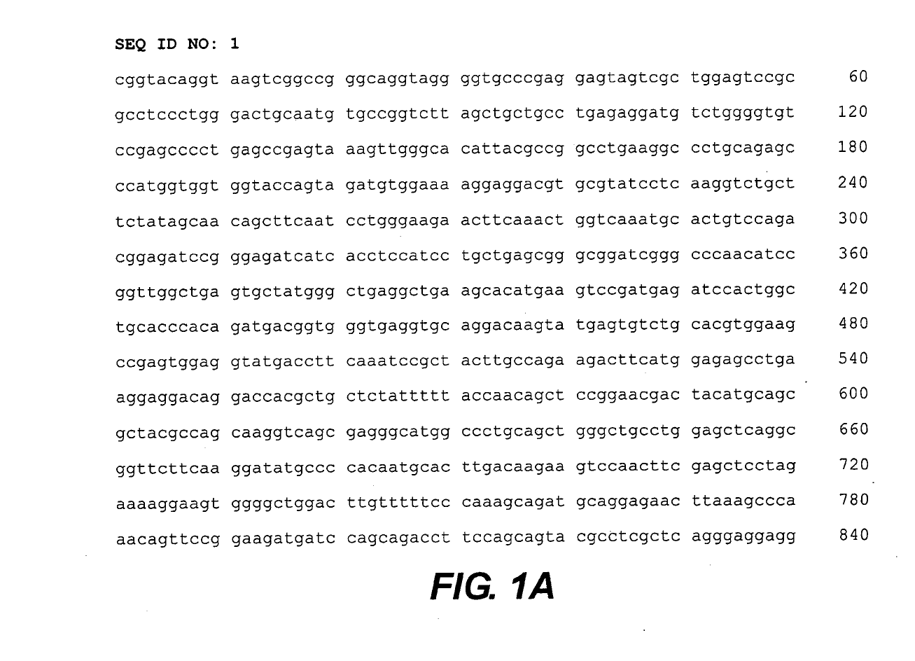

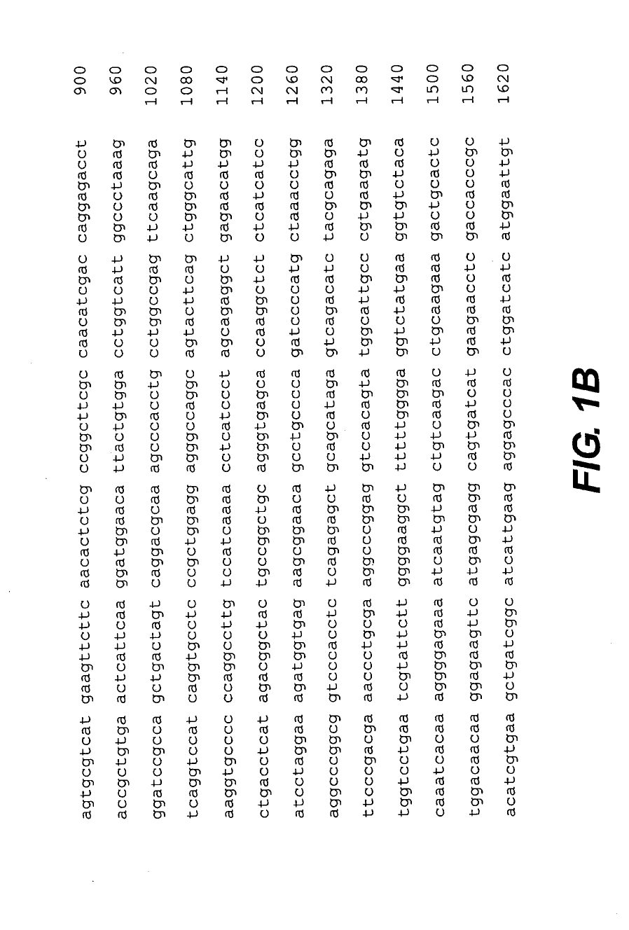

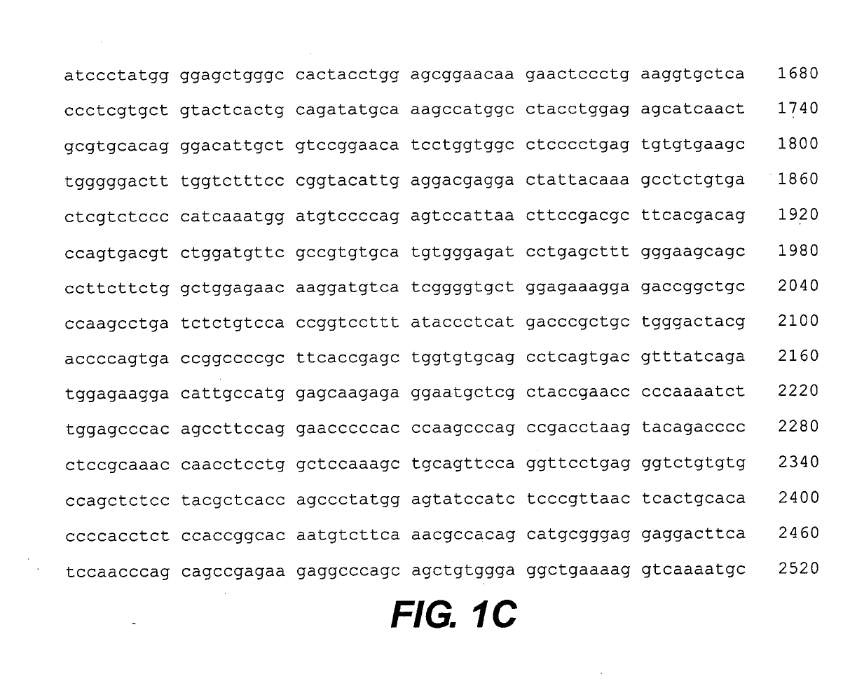

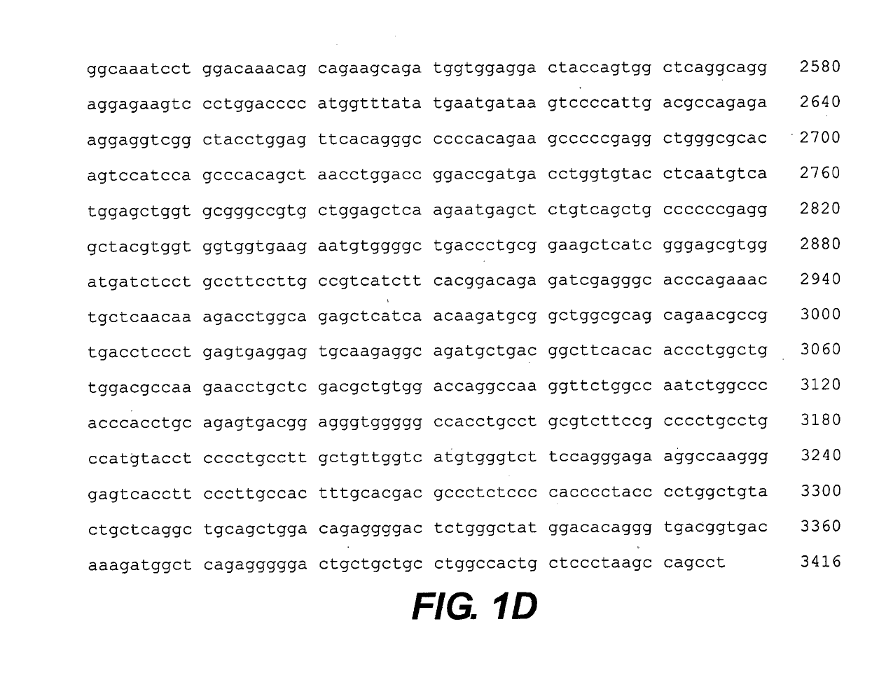

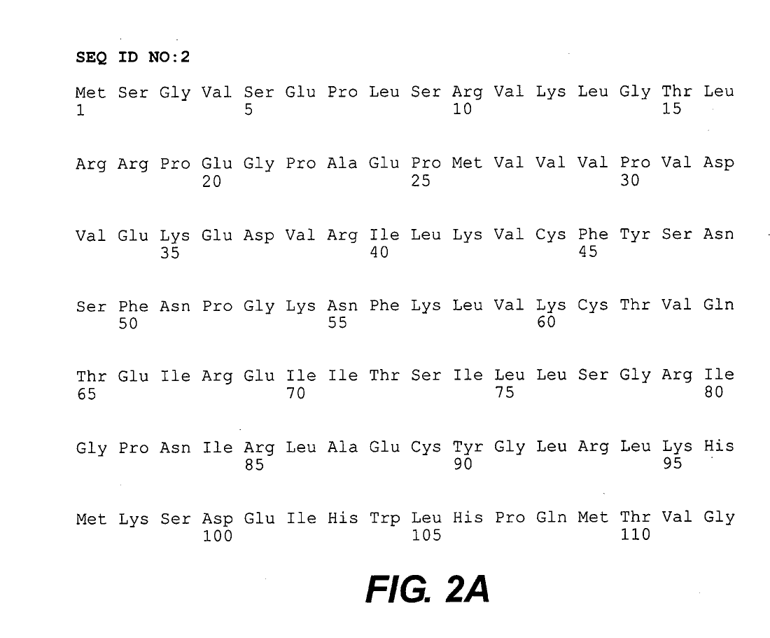

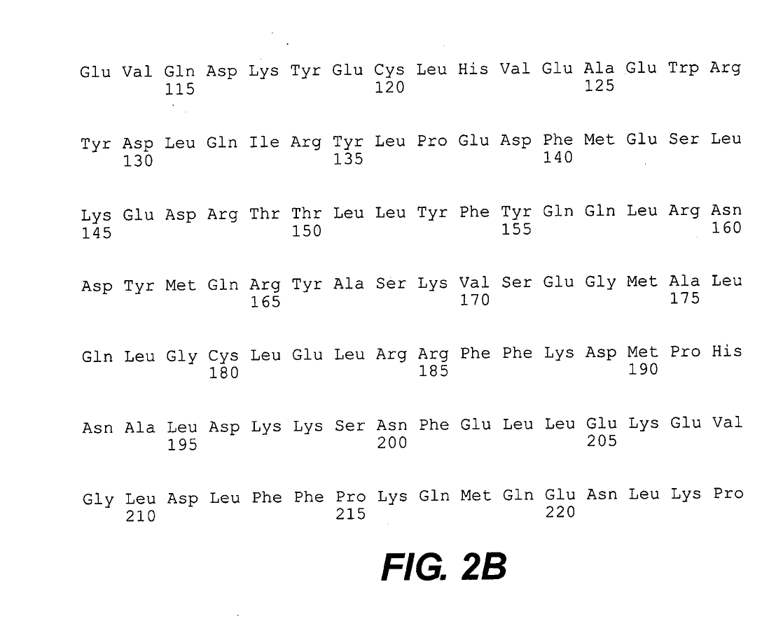

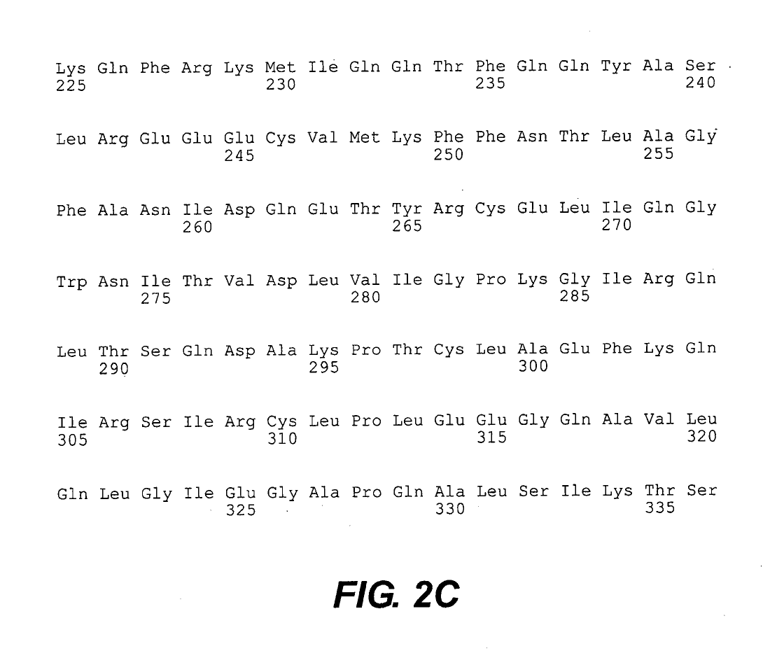

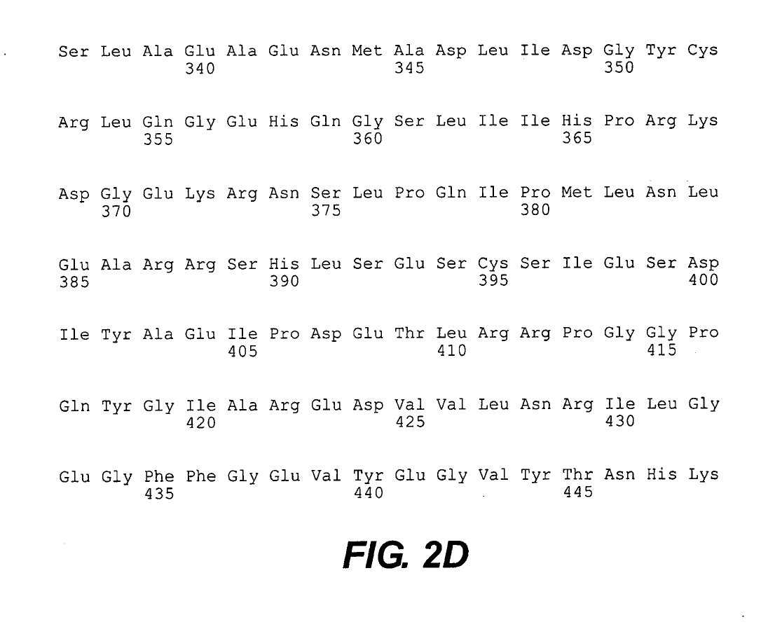

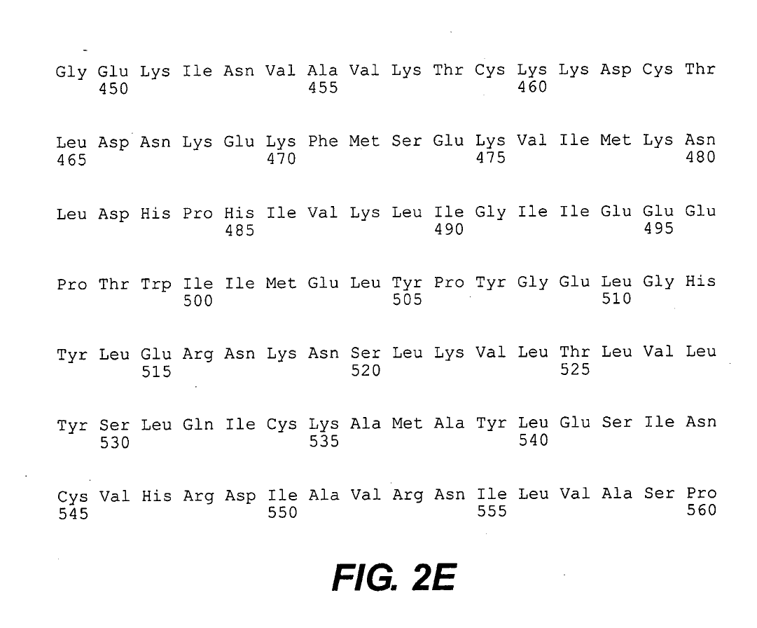

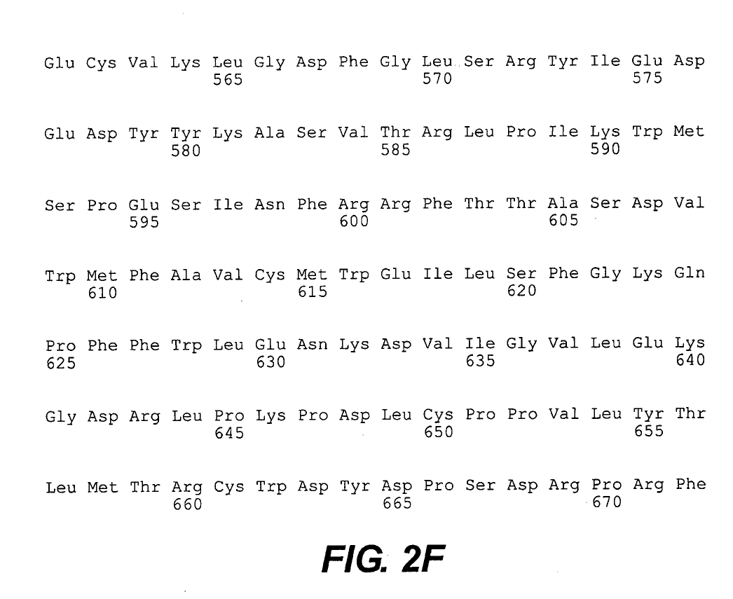

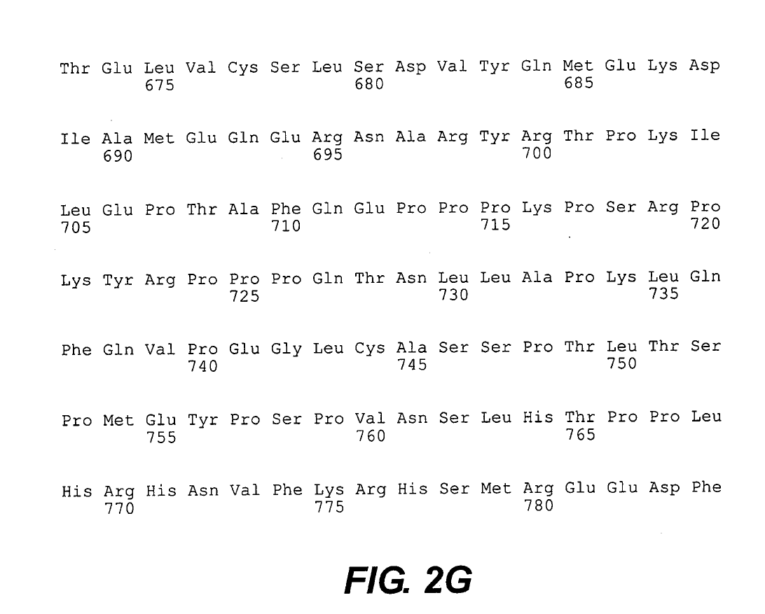

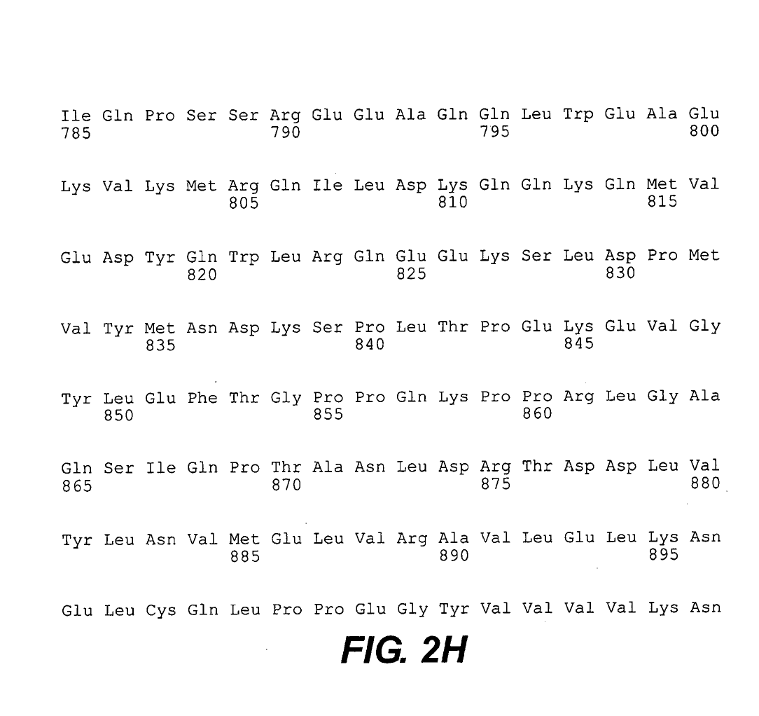

Pyk2 is a protein of 1,009 amino acids, containing a long amino-terminal sequence of 425 amino acids followed by a protein tyrosine kinase domain of 256 amino acids, two proline-rich domains, and a large carboxy-terminal region (See FIGS. 1A-D and 2A-I for the DNA sequence and amino acid sequence, respectively). The kinase domain of Pyk2 contains motifs conserved among protein tyrosine kinases, including a DFG tripeptide motif and an ATP-binding motif (GXGXXG), followed 17 amino acids downstream by the sequence, AXK (see FIG. 2A-1).

Pyk2 is activated by external stimuli that increase the intracellular concentration of calcium (Lev et al., 1995, Nature 376:737-745). Pyk2 becomes phosphorylated on tyrosine residues in response to such external stimuli. Tyrosine phosphorylation of Pyk2 is also stimulated by signals that activate a G-protein coupled receptor. For example, tyrosine phosphorylation of Pyk2 is induced by the neuropeptide bradykinin, which activates the B₂ G-protein-coupled receptor that stimulates the production of diacylglycerol (DAG) and inositol-1,4,5-triphosphate (Ins(1,4,5)P₃) by activating phospholipase C. Id. DAG, in turn, activates the serine/threonine protein kinase C (PKC), while Ins(1,4,5)P₃ triggers the release of calcium from intracellular stores. The increase in intracellular calcium concentration induces the tyrosine phosphorylation of Pyk2. Activation of Pyk2 by tyrosine phosphorylation in turn leads to tyrosine phosphorylation of a delayed rectifier-type potassium channel protein, suppressing potassium currents via this channel. Id. Thus, Pyk2 may be activated by and regulate the action of ion-channel proteins.

Pyk2 may also play a role in coupling external stimuli received by a cell to the regulation of transcriptional events inside the nucleus. Studies have shown that overexpression of Pyk2 leads to activation of the mitogen-activated protein (MAP) kinase signaling pathway, which leads to changes in transcription. Id. Thus, Pyk2 is thought to act as an intermediate protein that connects external stimuli to the regulation of gene expression.

In sum, Pyk2 may be an important protein coupling neuropeptides that activate G-protein-coupled receptors or neurotransmitters that stimulate calcium influx with downstream signaling events that regulate neuronal plasticity, cell excitability, and synaptic efficiency (Dikic et al., 1996, Nature 383:547-550). Therefore, modulators of Pyk2 may have general utility in preventing or inhibiting neurodegenerative diseases or conditions.

The present invention is intended to be used as either a stand-alone therapy, or as a conjunctive therapy with other agents that are either palliative (e.g., agents that relieve the symptoms of the disorder to be treated), and/or agents that target the etiology of the disorder. For example, the administration to a subject of a composition that modulates Pyk2 activity may be carried out in conjunction with the administration of L-DOPA, dopamine agonists, monoamine oxidase B inhibitors, or any other composition useful in the treatment of a neurodegenerative disease, such as Parkinson's disease.

Any method or compound that modulates Pyk2 activity is part of the invention. Specific agents and methods of the invention will now be discussed in more detail. These descriptions are exemplary of different embodiments of the present invention, and other agents and embodiments will be obvious to one skilled in the art upon reading the present disclosure. The present invention is intended to encompass these various embodiments as well those specifically described.

### Dominant Negative Pyk2

Derivatives of Pyk2 may be produced by techniques known in the art, including, but not limited to, amino acid deletions, additions, substitutions, or other manipulations that produce a molecule that possesses the property of interfering with the normal activity of Pyk2 and thereby preventing cell-death when expressed.

The engineering of dominant negative forms of proteins is routine and well known in the art. See, e.g., Sheppard, 1994, Am. J. Respir. Cell Mol. Biol. 11:1-6; Herskowitz, 1987, Nature 329:219-222. Briefly, a protein of interest may be analyzed at its DNA and/or amino acid level to determine regions important for protein function. For example, amino acid sequences may be compared in different species and conserved amino acids can be identified. These conserved amino acids are likely to be found in regions important for protein function. These regions may include, but are not limited to, regions important for catalytic activity of the protein, a ligand-binding domain of a receptor, a receptor-binding domain of a ligand, regions implicated in multimerization of the protein, and targets of phosphorylation. A phenotypically dominant negative protein may be produced without affecting protein expression by incorporating mutations or modifications within those regions. Mutations may include amino acid substitutions, deletions, or additions, or combinations thereof.

Mutation of a protein may be achieved following techniques that are routine in the art. Mutations may be introduced at the nucleic acid level at particular locations by synthesizing oligonucleotides containing a mutant sequence, flanked by restriction sites enabling ligation to fragments of the native sequence. Following ligation, the resulting reconstructed sequence contains the desired insertion, substitution, or deletion. See Sambrook et al., Molecular Cloning: A Laboratory Manual, Vols 1-3 (2d ed. 1989), Cold Spring Harbor Laboratory Press.

Alternatively, oligonucleotide-directed site-specific mutagenesis procedures may be employed to provide an altered nucleotide sequence wherein predetermined sequences may be altered by substitution, deletion or insertion. Exemplary methods of making the alterations set forth above are known in the art (Walder et al., 1986, Gene 42:133-139; Bauer et al., 1985, Gene 37:73-81; Smith et al., Genetic Engineering: Principles and Methods, (1981), Plenum Press; Kunkel, 1985, Proc. Natl. Acad. Sci. USA 82:488-492; Kunkel et al., 1987, Methods in Enzymol. 154:367-382; U.S. Pat. Nos. 4,518,584 and 4,737,462, all of which are incorporated by reference). Other methods known in the art may also be used.

Modifications may also be incorporated at the amino acid level. Modifications may include, for example, acetylation, acylation, ADP-ribosylation, amidation, covalent attachment of another functional moiety, covalent attachment of a lipid or lipid derivative, covalent attachment of phosphotidylinositol, cross-linking, cyclization, disulfide bond formation or cleavage, demethylation, formation of covalent cross-links, formylation, gamma-carboxylation, glycosylation, glycophosphatidylinositol (GPI) anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination. See, for instance, Creighton, Proteins—Structure and Molecular Properties, (2d ed. 1993), W. H. Freeman and Company, New York; Johnson, Post Translational Covalent Modification of Proteins, (1983) Academic Press, New York; Seifter et al., 1990, Meth. Enzymol. 182:626-646; Rattan et al., 1992, Ann. N.Y. Acad. Sci. 663:48-62. Preparation of these modified proteins may, for example, be useful if direct administration of the dominant negative protein, rather than administration of a nucleic acid encoding the dominant negative protein, is contemplated.

Regions in Pyk2 that may be modified to produce a dominant negative Pyk2 include, but are not limited to, the kinase domain and the FAT domain. For example, the DFG tripeptide motif, the ATP-binding motif (GXGXXG) and/or the AXK sequence conserved in the kinase domain may be modified. Whether any amino acid change (deletion, addition, substitution, modification, or a combination thereof) results in a dominant negative Pyk2 can readily be determined by assaying the cell-death prevention activity of the dominant negative Pyk2. Examples of assays are presented in Examples 1 and 2.

### Antisense Oligonucleotides

Antisense oligonucleotides are small nucleic acids which are complementary to the “sense” or coding strand of a given gene, and are thus able to stably and specifically hybridize with the RNA transcript of a gene. Uses of antisense oligonucleotides are known in the art. Holt et al., 1988, Mol. Cell Biol. 8:963-973, have shown that antisense oligonucleotides hybridizing specifically with RNA transcripts of the oncogene c-myc, when added to cultured HL60 leukemic cells, inhibit proliferation and induce differentiation. Anfossi et al., 1989, Proc. Natl. Acad. Sci. USA 86:3379-3383, have shown that antisense oligonucleotides specifically hybridizing with RNA transcripts of the c-myb oncogene inhibit proliferation of human myeloid leukemia cell lines. Wickstrom et al., 1988, Proc. Nat. Acad. Sci. USA 85:1028-1032, have shown that expression of the protein product of the c-myc oncogene as well as proliferation of HL60 cultured leukemic cells are inhibited by antisense oligonucleotides hybridizing specifically with c-myc mRNA. Moreover, Higgins et al., 1993, Proc. Nat. Acad. Sci. USA 90:9901-9905, demonstrated tumor regression in vivo by administering dexamethasone-inducible antisense RNA to p65 into tumor-bearing nude mice. Kitajima et al., 1992, Science 258:1792-1795, observed growth inhibition of transplanted fibrosarcomas in mice after administration of antisense oligonucleotides to NF-kappa B. Li et al., 2002, Clin. Cancer Res. 8:3570-3578, have shown that administration of an antisense oligonucleotide to epidermal growth factor receptor (EGFR) in squamous cell carcinoma tumor-bearing mice partially inhibited tumor growth. In another example, Rijcken et al., 2002, Gut 51:529-535, have shown that administration of antisense oligonucleotides directed against the intercellular adhesion molecule 1 (ICAM-1) or vascular cell adhesion molecule 1 (VCAM-1) into Sprague-Dawley rats significantly reduced leucocyte adhesion to endothelial cells and also significantly decreased inflammation. Li et al. therefore suggest that ICAM-1 and VCAM-1 antisense oligonucleotide therapy may be useful for anti-inflammatory treatment in inflammatory bowel disease.

Antisense oligonucleotides of the present invention specifically hybridize with selected DNA or RNA derived from a Pyk2 gene. The invention also provides oligonucleotides for selective inhibition of expression of Pyk2. “Targeting” an oligonucleotide to a chosen nucleic acid target, in the context of this invention, may be a multistep process. The process usually begins with identifying a nucleic acid sequence whose function is to be modulated. This may be, for example, a cellular gene (or mRNA made from the gene) whose expression is associated with a particular disease state, or a foreign nucleic acid from an infectious agent. In the present invention, the target is a nucleic acid encoding Pyk2; in other words, the Pyk2 gene or RNA expressed from the Pyk2 gene. The target may comprise the 5′ or 3′ untranslated nucleic acid region of Pyk2. The targeting process also includes determination of a site or sites within the nucleic acid sequence for the oligonucleotide interaction to occur such that the desired effect, i.e., modulation of gene expression, will result. Once the target site or sites have been identified, oligonucleotides are chosen which are sufficiently complementary to the target, i.e., hybridize sufficiently well and with sufficient specificity, to give the desired modulation.

In the context of this invention “modulation” means either inhibition or stimulation. This modulation can be measured in ways that are routine in the art, for example by Northern blot assay of mRNA expression or by Western blot assay of protein expression. Effects on neurotoxicity or neuronal cell death can also be measured, as taught in the examples of the instant application.

“Hybridization”, in the context of this invention, means hydrogen bonding, also known as Watson-Crick base pairing, between complementary bases, usually on separate nucleic acid strands or on two regions of a single nucleic acid strand. Guanine and cytosine are examples of complementary bases that are known to form three hydrogen bonds between them. Adenine and thymine are examples of complementary bases that form two hydrogen bonds between them. “Specifically hybridizable” and “complementary” are terms that indicate a sufficient degree of complementarity such that stable and specific binding occurs between the DNA or RNA target and the oligonucleotide. It is understood that an oligonucleotide need not be 100% complementary to its target nucleic acid sequence to be specifically hybridizable.

While it is understood that the lower the salt concentration, the higher the stringency, in general, hybridization conditions are based on the melting temperature (Tm) of the nucleic acid binding complex or probe at a given ionic strength and are typically classified by the degree of stringency of the conditions under which hybridization is measured. For example, maximum stringency typically occurs at about Tm-5° C. (5° C. below the Tm of the probe) with salt concentrations in the washing solution typically being 0.1×SSC (20×SSC=3.0 M NaCl/0.3 M trisodium citrate). High stringency typically occurs at about 5-10° C. below the Tm; intermediate stringency at about 10-20° C. below the Tm of the probe; and low stringency at about 20-25° C. below the Tm. See, e.g., U.S. Pat. Nos. 6,051,385 and 6,111,090. Functionally, maximum stringency conditions may be used to identify nucleic acid sequences having identity or near-identity with the hybridization probe; while high stringency conditions may be used to identify nucleic acid sequences having about 80% or more sequence identity with the probe. Persons skilled in the art will appreciate that specific conditions will vary according to the composition of the probe, composition of the target substrate, and the like. An example of high stringency conditions for a probe having a Tm of 70° C. includes hybridization at about 65° C. in about 5×SSPE and washing at about 65° C. in about 0.1×SSPE (where 1×SSPE=0.15 M sodium chloride, 0.010 M sodium phosphate, and 0.001 M disodium EDTA). Examples of various conditions of stringency are taught in Wahl and Berger, 1987, Methods Enzymol. 152:399-407 and Kimmel, 1987, Methods Enzymol. 152:507-511. One general guide for nucleic acid hybridization is Tijssen, Laboratory Techniques in Biochemistry and Molecular Biology, Hybridization with Nucleic Acid Probes, Part I Overview of Principles of Hybridization and the Strategy of Nucleic Acid Assays, Vol. 24 (1993), Elsevier.

An oligonucleotide is also specifically hybridizable when binding of the oligonucleotide to the target interferes with the normal function of the target molecule to cause a loss of utility, and there is a sufficient degree of complementarity to avoid non-specific binding of the oligonucleotide to non-target sequences under conditions in which specific binding is desired, i.e., under physiological conditions in the case of in vivo assays or therapeutic treatment or, in the case of in vitro assays, under conditions in which the assays are conducted.

In vitro or in vivo effects of an antisense oligonucleotide may be predicted by calculating the Tₘ, free energy change (ΔG°) involved in hybridization (ΔG°_{hyb}), in a structural change in the target nucleic acid from an optimal folding structure to a suboptimal one (ΔG°_{sc}), and in unfolding of the antisense oligonucleotide hairpin (ΔG°ₕₚ), if any. These values may be calculated according to Sugimoto et al., 2001, Current Medicinal Chemistry—Anti-Cancer Agents 1:95-112. In general, the antisense effect correlates with all of the above factors. Specifically, the higher the Tₘ and the more favorable the ΔG°_{hyb}, the greater the effect of the antisense oligonucleotide. Similarly, the more favorable the ΔG°ₕₚ, the greater the effect of the antisense oligonucleotide. However, if ΔG°_{sc} indicates that the target nucleic acid may fold undesirably, the antisense oligonucleotide may not bind efficiently and may have less of an effect on the target molecule.

In the context of this invention, the term “oligonucleotide” refers to an oligomer or polymer of nucleotides or nucleotide monomers comprising naturally occurring bases, sugars and intersugar (backbone) linkages. The term “oligonucleotide” also includes oligomers comprising non-naturally occurring monomers, or portions thereof, which function similarly. Such modified or substituted oligonucleotides are often preferred over native forms because of properties such as, for example, enhanced cellular uptake and increased stability in the presence of nucleases.

The antisense oligonucleotides used in this invention may be conveniently and routinely made through the well-known technique of solid phase synthesis. Equipment for such synthesis is sold by several vendors including Applied Biosystems (Foster City, Calif.). Any other means for such synthesis may also be employed; the actual synthesis of the oligonucleotides is well within the talents of those of ordinary skill in the art. It is also well known to use similar techniques to prepare other oligonucleotides such as the phosphorothioates and alkylated derivatives (Zon, 1988, Pharm. Res. 5:539-549). It is also well known to use similar techniques and commercially available modified amidites and controlled-pore glass (CPG) products such as biotin, fluorescein, acridine or psoralen-modified amidites and/or CPG (available from Glen Research, Sterling Va.) to synthesize fluorescently labeled, biotinylated or other modified oligonucleotides such as cholesterol-modified oligonucleotides.

The antisense oligonucleotides of the invention will generally be at least about 7, usually at least about 12, more usually at least about 20 nucleotides in length, and not more than about 500, usually not more than about 50, more usually not more than about 35 nucleotides in length, where the length is governed by efficiency of inhibition, specificity, including absence of cross-reactivity, and the like. It has been found that short oligonucleotides, of from 7 to 8 bases in length, can be strong and selective inhibitors of gene expression. See Wagner et al., 1996, Nature Biotechnol. 14:840-844. In the context of this invention, it is understood that this encompasses naturally and non-naturally occurring oligomers as hereinbefore described.

Antisense molecules may also be produced by in vivo expression of all or a part of the target gene sequence in an appropriate vector, where the transcriptional initiation is oriented such that an antisense strand is produced as an RNA molecule. The RNA antisense sequence is complementary to the RNA of the targeted gene, and inhibits expression of the targeted gene products. Antisense molecules inhibit gene expression through various mechanisms, e.g. by reducing the amount of mRNA available for translation, through activation of RNAse H, or steric hindrance.

Alternatively, antisense molecules may be produced by in vitro transcription of all or part of the target gene sequence from an appropriate vector, as described above, such that an antisense strand is produced as an RNA molecule. Synthesis of single-stranded RNA in vitro has been facilitated by the development of plasmid vectors containing polycloning sites downstream from powerful promoters derived from the Salmonella typhimurium bacterophage SP6 (Green et al., 1983, Cell 32:681) or from the E. coli bacterophages T7 and T3 (Studier and Rosenberg, 1981, J. Mol. Bio. 153:503; Davanloo et al., 1984, Proc. Natl. Acad. Sci. USA 81:2035; Tabor and Richardson, 1985, Proc. Natl. Acad. Sci. USA 82:1074). The DNA-dependent RNA polymerases encoded by their respective bacteriophages specifically recognize their cognate promoters and do not use promoters recognized by other polymerases, such as other bacteriophage, bacterial or eukaryotic promoters present in a plasmid vector. Thus, when a linearized plasmid is incubated in vitro with the appropriate DNA-dependent RNA polymerase and the four rNTPs (ribonucleotide triphosphates), virtually all RNA synthesis is initiated at the select bacteriophage promoter.

In vitro transcription may be performed by any of the methods known in the art. See Sambrook et al., Molecular Cloning: A Laboratory Manual, Vols 1-3 (2d ed. 1989), Cold Spring Harbor Laboratory Press; Miligan et al., 1987, Nucl. Acids Res. 15:8783; Milligan and Uhlenbeck, 1989, Meth. Enzymol. 180:51. Any of the commercially available kits may also be used, e.g., Riboprobe® In Vitro Transcription Systems from Promega (Madison, Wis.).

### Interfering RNA (RNAi)

RNAi is a partially or fully double-stranded RNA molecule similar to a portion of a target nucleic acid sequence. RNAi triggers a cellular process when it enters a cell and causes the degradation of not only the invading RNAi molecule itself, but also of the single-stranded (ssRNAs) RNAs of identical sequences, including endogenous mRNAs. For a review, see e.g., Nishikura, 2001, Cell 107:415-418; Hannon, 2002, Nature 4418:244-251. The general method of making and using RNAi is disclosed in U.S. Pat. No. 6,506,559.

The target nucleic acid may be a gene derived from the cell (i.e., a cellular gene), a transgene (i.e., a gene construct inserted at an ectopic site in the genome of the cell), or a gene from a pathogen capable of infecting an organism from which the cell is derived.

In one embodiment, RNAi containing a nucleotide sequence 100% identical to a portion of the target gene may be used. In another embodiment, the RNAi has greater than 90% sequence identity with a portion of the target gene. In a further embodiment, RNAi with greater than 80%, 70%, 60% or 50% sequence identity with a portion of the target gene may be used. Sequence identity may be optimized by sequence comparison and alignment algorithms known in the art and calculating the percent difference between the nucleotide sequences by, for example, the Smith-Waterman algorithm as implemented in the BESTFIT software program using default parameters (e.g., University of Wisconsin Genetic Computing Group). The length of the identical nucleotide sequences may be at least 25, 50, 100, 200, 300 or 400 bases.

The RNAi may comprise one or more strands of polymerized ribonucleotide. RNAi may include modifications to either the phosphate-sugar backbone or the nucleoside. The double-stranded structure may be formed by a single self-complementary RNA strand or two complementary RNA strands. RNA duplex formation may be initiated either inside or outside the cell. The RNA strands need not be polyadenylated and the RNA strands need not be capable of being translated into a polypeptide by a cell's translational apparatus. The RNA may be synthesized either in vivo or in vitro as described above for antisense oligonucleotides. Alternatively, RNA may be chemically or enzymatically synthesized by manual or automated reactions.

In one embodiment, RNAi may be synthesized in vitro and introduced into cells by methods known in the art to transiently suppress gene expression. RNAi synthesized in vitro by chemical methods may be from about 19 to about 23 nucleotides long. RNAi synthesized in vitro by enzymatic methods may be any length. In another embodiment, RNAi may be expressed in vivo from a vector.

### Other Inhibitors of Pyk2

Pyk2 inhibitors of the invention also encompass chemicals, naturally-occurring, recombinant, or synthetic peptides, polypeptides, proteins, polysaccharides, small molecules and other compounds designed to reduce or inhibit Pyk2 activity in a cell or in a host. These Pyk2 inhibitors may, for example, reduce or inhibit Pyk2 activity by interfering with a pathway that leads to activation of Pyk2, or, for example, by interfering with a pathway that is directly regulated by an activated Pyk2. The Pyk2 inhibitors of the invention may also reduce or inhibit Pyk2 activity by, for example, directly binding to Pyk2 or by, for example, preventing phosphorylation of Pyk2.

Peptides, polypeptides, and proteins that inhibit Pyk2 activity may be generated according to methods known in the art. For example, phage peptide display libraries can be used to express large numbers of peptides that can be screened in vitro to identify peptides that specifically bind Pyk2 or inhibit Pyk2 activity. Phage display technology provides a means for expressing a diverse population of random or selectively randomized peptides. Various methods of phage display and methods for producing diverse populations of peptides are well known in the art. For example, Ladner et al. (U.S. Pat. No. 5,223,409), describes methods for preparing diverse populations of binding domains on the surface of a phage. Ladner et al. describe phage vectors useful for producing a phage display library, as well as methods for selecting potential binding domains and producing randomly or selectively mutated binding domains. Screening of a phage display library generally involves in vitro panning of the library using a purified target molecule. Phage that bind the target molecule can be recovered, individual phage can be cloned and the peptide expressed by a cloned phage can be determined.

Similarly, Smith and Scott, 1993, Meth. Enzymol. 217:228-257 and 1990, Science 249:386-390, describe methods of producing phage peptide display libraries, including vectors and methods of diversifying the population of peptides that are expressed. See also, WO 91/07141 and WO 91/07149. Phage display technology can be particularly powerful when used, for example, with a codon based mutagenesis method, which can be used to produce random peptides or randomly or desirably biased peptides. See, e.g., U.S. Pat. No. 5,264,563. These and other well known methods can be used to produce a phage display library, which can be subjected to an in vitro panning method in order to identify a peptide, polypeptide, or protein that binds to Pyk2.

Peptides, polypeptides, proteins, polysaccharides, and the like that bind Pyk2 or inhibit Pyk2 activity may also be isolated from natural sources, and then optionally processed (e.g., via peptide cleavage) or, alternatively, synthesized by conventional techniques known in the art such as solid phase synthesis or recombinant expression. See, e.g., See Sambrook et al., Molecular Cloning: A Laboratory Manual, Vols 1-3 (2d ed. 1989), Cold Spring Harbor Laboratory Press. Automatic peptide synthesis can be performed using commercially available apparatus from manufacturers such as Applied Biosystems (Foster City, Calif.), and methods of doing so are well established. Recombinant production of the proteins may be in prokaryotic, such as phage or bacterial cells or eukaryotic systems, such as yeast, insect, or mammalian cells. Alternatively, proteins can be produced using cell-free in vitro systems known in the art.

A peptide, polypeptide, or protein that binds to Pyk2 or inhibits Pyk2 activity may be expressed as a fusion protein with a heterologous peptide. The peptide, polypeptide, or protein of the invention may be linked at its amino terminus, its carboxyl terminus, or both to a heterologous peptide. Optionally, multiple repeats of the heterologous peptide can be present in the fusion protein. Optionally, a peptide, polypeptide, or protein of the invention may be linked to multiple copies of a heterologous peptide, for example, at both the N and C termini of the heterologous peptide. Some heterologous proteins serve to enhance the half-life of the fused peptide, polypeptide, or protein, thereby increasing therapeutic efficacy in vivo. See, e.g., U.S. Pat. Nos. 5,876,969 and 5,565,335.

Other Pyk2 inhibitors may be identified by screening for chemicals, naturally-occurring, recombinant, or synthetic peptides, polypeptides, proteins, or polysaccharides for their ability to inhibit Pyk2 activity. For example, chemicals that may inhibit the phosphorylation by Pyk2 may include tyrphostins, quinazolines, quinaxolines, and quinolines. These are well known in the art.

Candidates for Pyk2 inhibitors including chemicals, naturally-occurring, recombinant or synthetic peptides, polypeptides, proteins, or polysaccharides may be screened for inhibition of Pyk2 activity according to the method disclosed in the Examples. Additionally, a Pyk2 inhibitor may be screened for inhibition of phosphorylation of Pyk2, induction of dephosphorylation of Pyk2, binding to Pyk2, or interference with a pathway leading to Pyk2 activation or with a pathway directly regulated by activated Pyk2.

### Vectors Expressing a Pyk2 Modulator

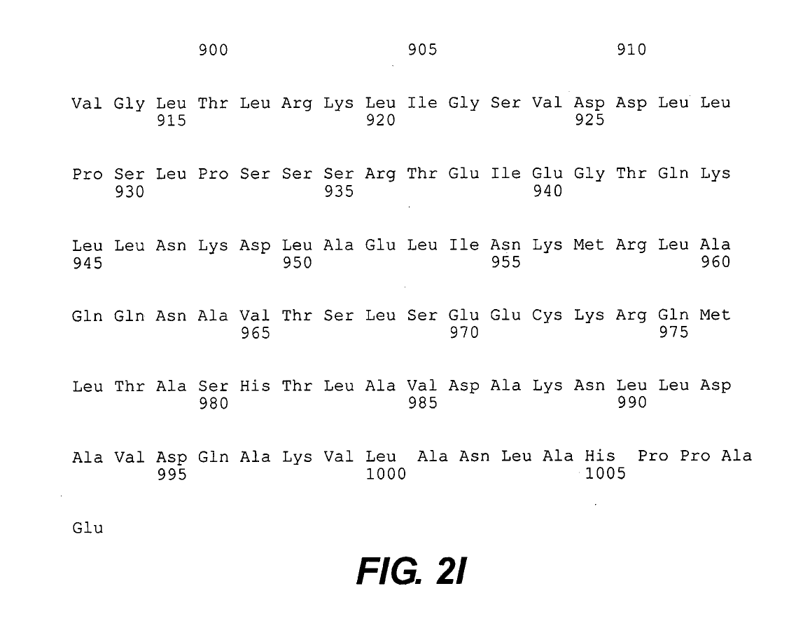

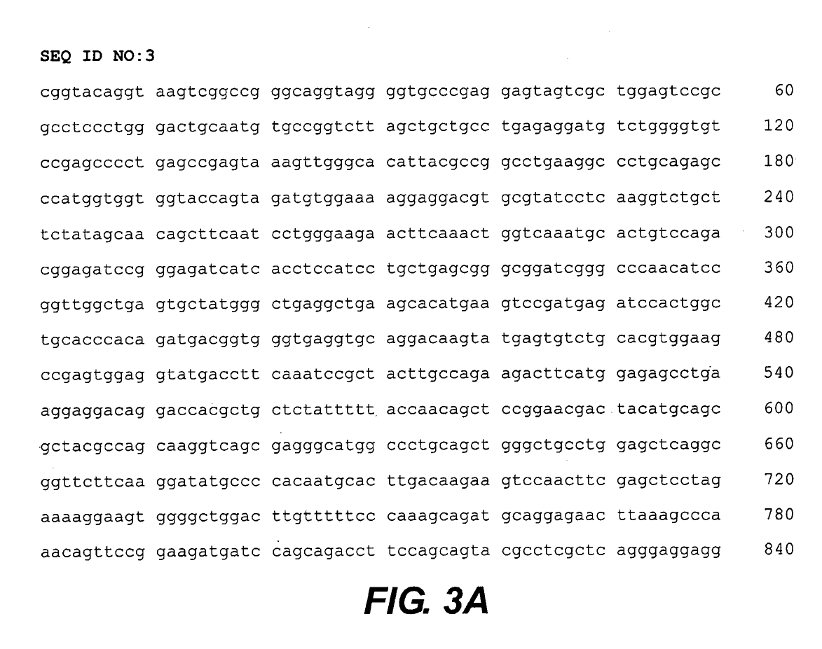

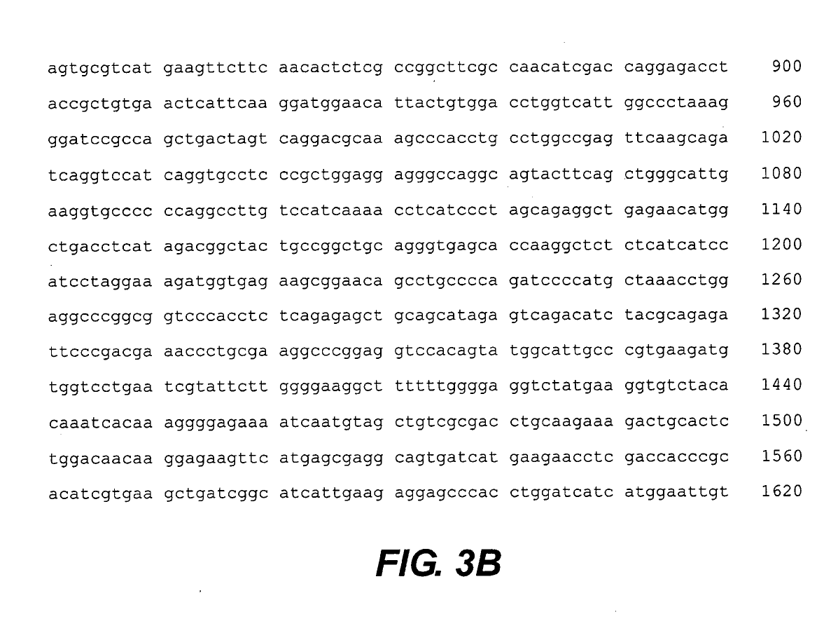

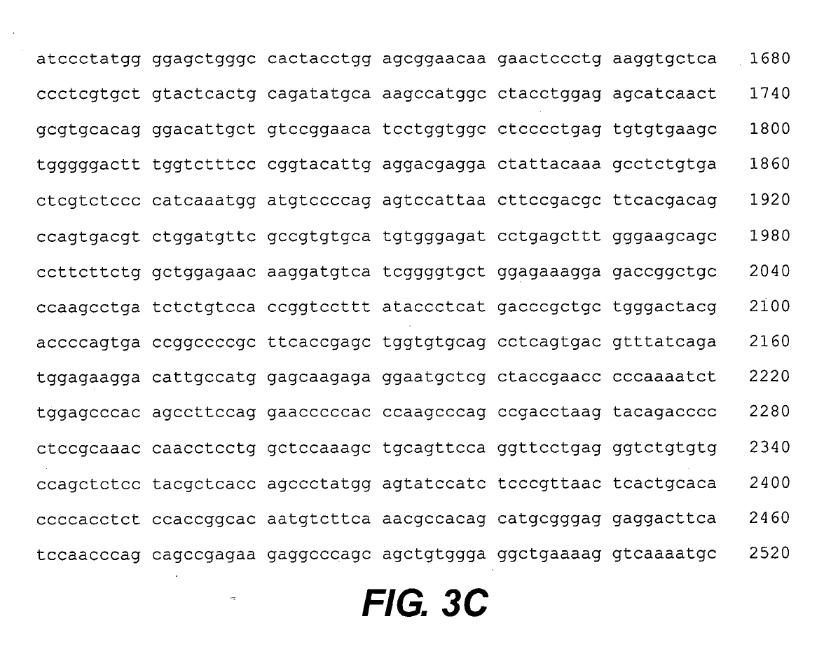

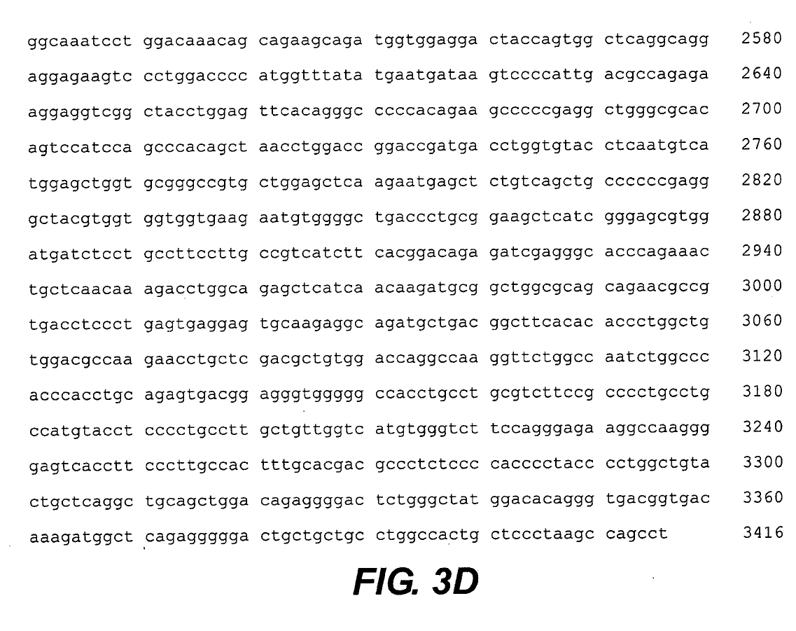

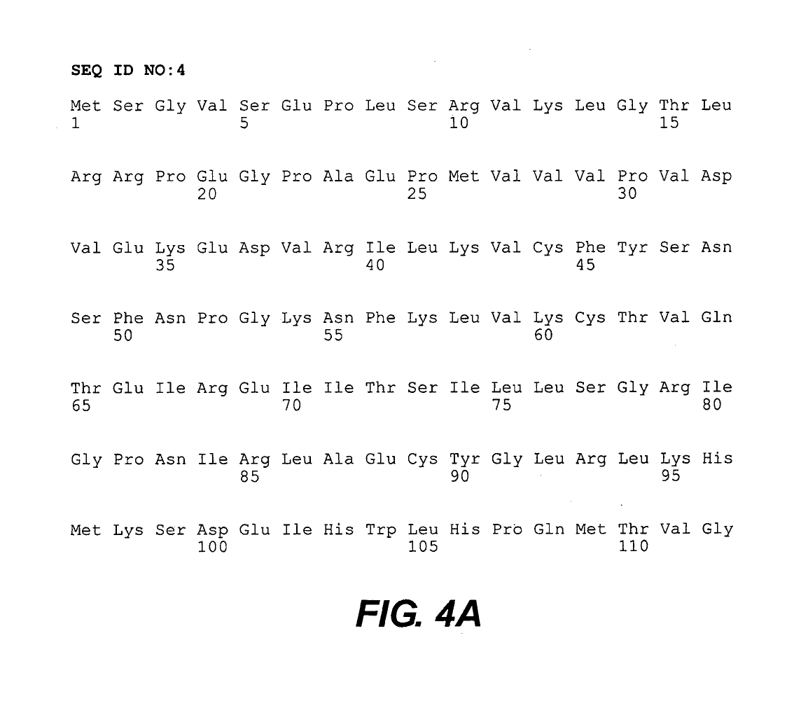

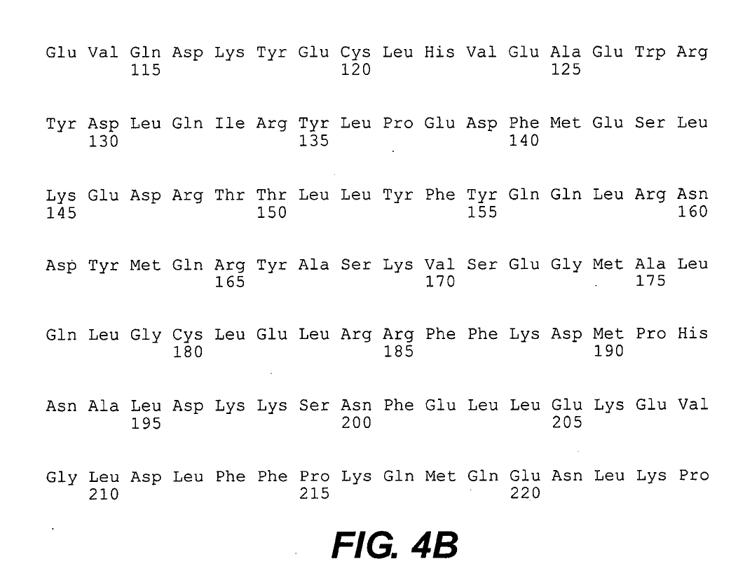

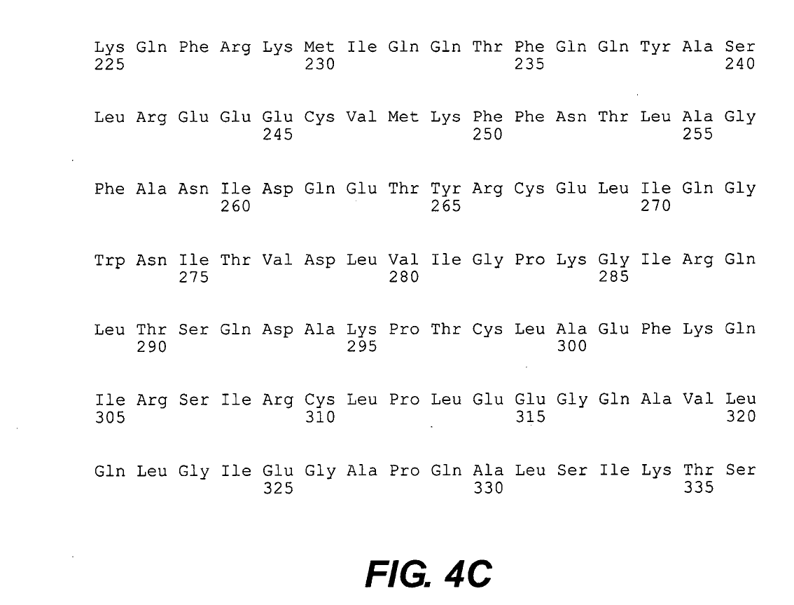

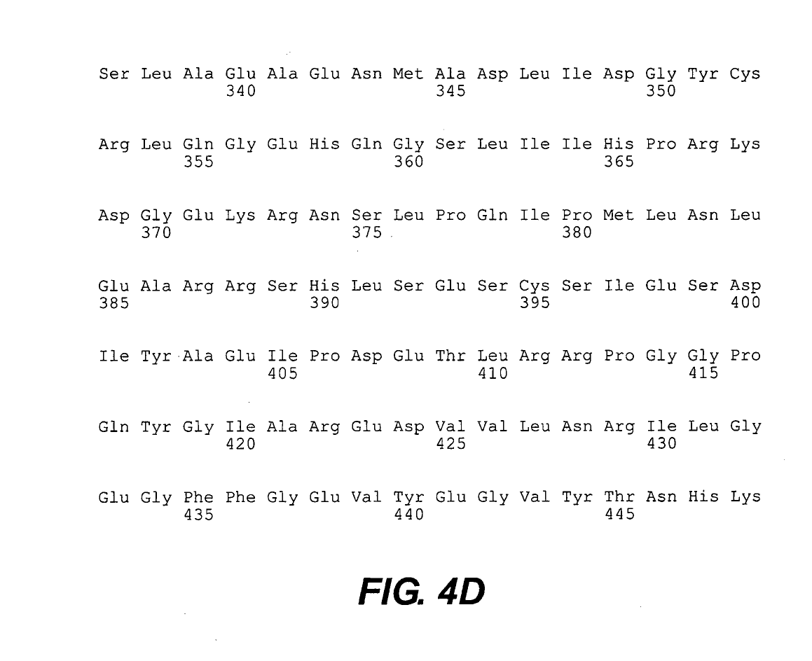

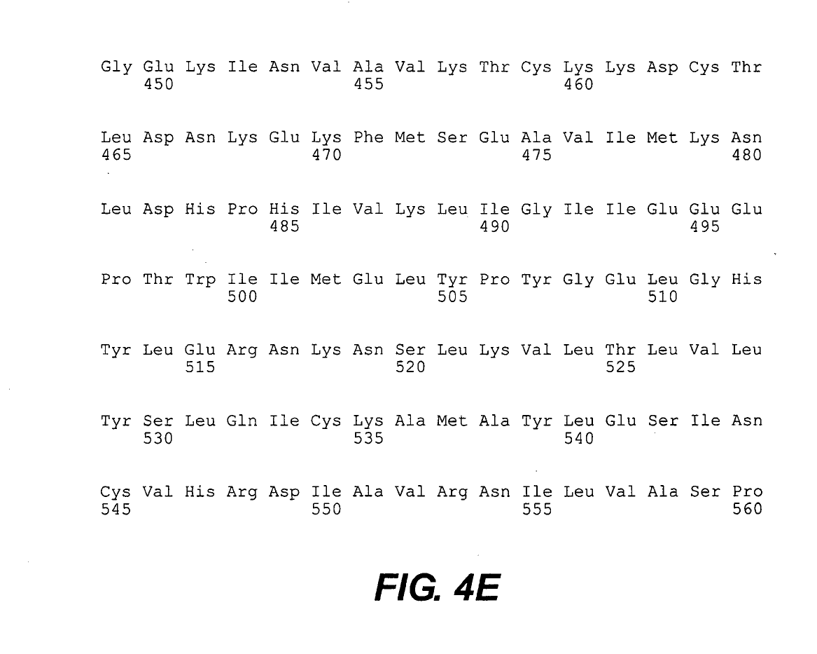

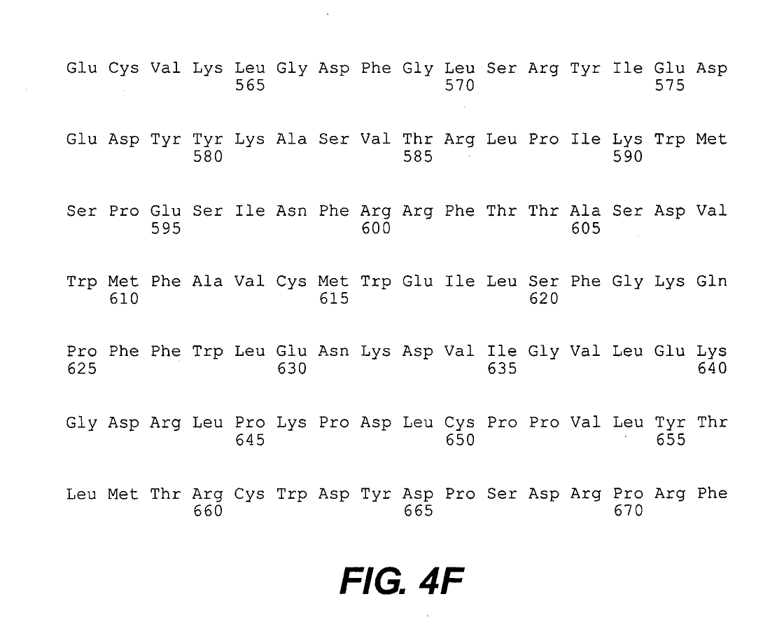

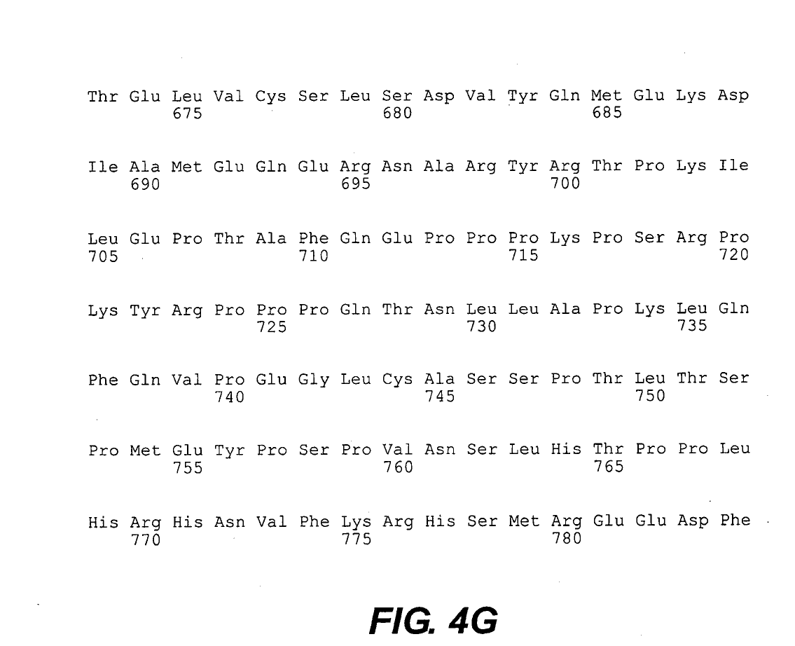

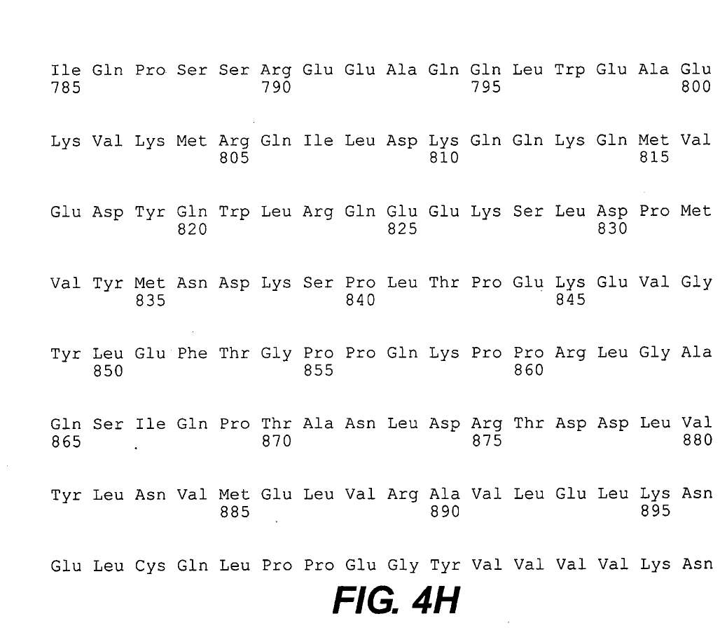

The DNA sequence and amino acid sequence of human Pyk2 are shown in FIGS. 1A-D and 2A-I, respectively. Moreover, the DNA sequence and amino acid sequence of a dominant-negative human Pyk2 are shown in FIGS. 3A-D and 4A-I, respectively. Construction of an expression vector comprising a nucleic acid encoding a Pyk2 inhibitor linked to one or more transcriptional regulatory sequences may be accomplished by techniques known to those skilled in the art and the use of appropriate vectors.

An appropriately constructed expression vector may contain, for example, an origin of replication for autonomous replication in the host cells, one or more selectable markers, a limited number of useful restriction enzyme sites, a potential for high copy number, and one or more active promoters. Expression vectors may originate from a variety of sources such as viruses, plasmids, or the cells of a higher organism, such as yeast and mammalian cells. The vectors of the invention may be introduced in vitro for in vitro testing of Pyk2 activity. Methods of introducing the vectors include viral-based approaches and nonviral approaches, such as lipofection, ligand-DNA conjugates and direct injection of naked DNA. See, e.g., U.S. Pat. No. 6,140,484. Other methods described herein or known in the art may be used.

The vectors of the invention may also be introduced ex vivo, where the cells to be transfected are first removed from the subject to be treated and cultured. The vectors of the invention are then introduced into the cultured cells in vitro by any of the methods described herein, or by other methods known in the art. The cells are then transplanted or administered back into the body. Alternatively, the vectors of the invention may be introduced in vivo.

In the case of virus-mediated gene transfer, host cells are transfected with vectors of the present invention by infection with mature virions containing hybrid vectors (the nucleic acid sequences of the invention along with selected viral sequences). The virions used to transfect host cells are preferably replication-defective, so that the virus is not able to form infectious virus particles in the host cells.

The virions may be produced by co-infection of cultured host cells with a helper virus. Following coinfection, the virions are isolated (e.g., by cesium chloride centrifugation) and any remaining helper virus is inactivated (e.g., by heating). The resulting mature virions contain a vector of the present invention and may be used to infect host cells in the absence of helper virus. Alternatively, high titers of replication-defective recombinant virus, free of helper virus, may be produced in packaging cell lines containing those components for which the virus is defective (Palu et al., 2000, Rev. Med. Virol. 10:185-202; Miller, 1990, Hum. Gene Ther. 1:5).

Several types of viruses, including retroviruses, adenoassociated virus (AAV), herpes virus, vaccinia virus, and several RNA viruses may be amenable for use as vectors in the present invention. Each type of virus has advantages and disadvantages, which are appreciated by those of skill in the art. For example, retroviral and AAV vectors may be more suitable for stable transfection than are adenovirus, vaccinia virus, or polio virus vectors. Methods for manipulating viral vectors are also known in the art. See, e.g., U.S. Pat. No. 6,140,484.

Retroviruses, like adenoassociated viruses, stably integrate their DNA into the chromosomal DNA of the target cell. Unlike adenoassociated viruses, however, retroviruses typically require replication of the target cells in order for proviral integration to occur. Accordingly, successful gene transfer with retroviral vectors depends on the ability to at least transiently induce proliferation of the target cells.

Retroviral vectors are attractive in part due to the efficiency of transfection—some vectors can stably transduce close to 100% of target cells. The use of retroviral vectors for in vivo gene therapy has been limited, in part, by the requirement of appropriate viral receptors on the target cell. Because the identities of most retroviral receptors are unknown, it has not been possible to determine the distribution of receptors in different cell types. Accordingly, the targeting of specific cell types by retroviral vectors has in many cases proven problematic. However, this difficulty may be circumvented by modifying the envelope protein of the retrovirus to contain a ligand for a known endogenous (not necessarily viral) receptor expressed on the target cells. An application of this technique is described in detail by Kasahara et al., 1994, Science 266:1373. The virus may also contain an unmodified envelope protein to facilitate cell entry.

Adenoassociated viruses are capable of efficiently infecting nondividing cells and expressing large amounts of gene product. Furthermore, the virus particle is relatively stable and amenable to purification and concentration (Smith-Arica et al., 2001, Curr. Cardiol. Rep. 3:43-49).

Replication-defective adenoviruses lacking portions of the E1 region of the viral genome may be propagated by growth in cells engineered to express the E1 genes. Most of the currently-used adenovirus vectors carry deletions in the E1A-E1B and E3 regions of the viral genome. A number of studies using adenoviral vectors have demonstrated that the vectors are efficient at transforming a significant fraction of cells in vivo, and that vector-mediated gene expression can persist for significant periods of time. See, e.g., Wilson, 2002, Curr. Pharm. Biotechnol. 3:151-164; Shimada et al., 2001, Surg. Today 31:597-604; Herman et al., 1999, Human Gene Ther. 10:1239-1249. Adenovirus vectors have also been used to deliver genes into the CNS, and transgene expression was demonstrated over widespread areas of the brain, even 12 months after injection of the adenovirus vectors (Zermansky et al., 2001, Mol. Ther. 4:490-498).

Herpes virus vectors are also well suited for the delivery and expression of foreign DNA in cells of the central nervous system (CNS), since they can efficiently infect mature, postmitotic neurons. Methods for manipulating the vectors and transfecting CNS cells are well known in the art. See, e.g., U.S. Pat. No. 6,140,484 for various references. Studies utilizing direct injection of vectors into CNS tissue have also been performed. See, e.g., Agudo et al., 2002, Hum. Gene Ther. 13:665-674; Zhang et al., 1992, Neuroreport 3:700-102.

In general, the expression vector may be introduced into a host cell via any one of a number of techniques including, but not limited to, transformation, transfection, infection, protoplast fusion, and electroporation. These methods may be used as required in in vitro or in vivo procedures.

Plasmids or vectors of the present invention may also be purified and injected directly into a target tissue, using naked DNA injection. Further, liposomes may be employed to deliver genes to target tissues using methods known in the art. See, e.g., U.S. Pat. Nos. 5,208,036, 5,264,618, 5,279,833 and 5,283,185. The liposomes may be constructed to contain a targeting moiety or ligand, such as an antigen, an antibody, or a virus on their surface to facilitate delivery to the appropriate tissue. The liposomes may also be surface-coated, e.g., by incorporation of phospholipid-polyethyleneglycol conjugates, to extend blood circulation time and allow for greater targeting via the bloodstream.

Receptor-mediated endocytic pathways for the uptake of DNA may permit the targeted delivery of genes to specific cell types in vivo. Receptor-mediated methods of gene transfer involve the generation of complexes between plasmid DNA and specific polypeptide ligands that can be recognized by receptors on the cell surface. See, e.g., Gupta et al., 2001, Gene Ther. 8:586-592; Wu et al., 1991, J. Biol. Chem. 266:14338-14342. One of the problems with receptor-mediated uptake for gene delivery is that the endocytic vesicles formed during this process may be transported to the lysosome, where the contents of the endosome are degraded. Methods have been developed to facilitate escape of the DNA from the endosome during the course of its transport. For example, either whole adenovirus (Wagner et al., 1992, Proc. Natl. Acad. Sci. USA 89:6099-6103) or fusogenic peptides of the influenza HA gene product (Wagner et al., 1992, Proc. Natl. Acad. Sci. USA 89:7934-7938) may be used to induce efficient disruption of DNA-containing endosomes.

In cases such as those outlined above, where a Pyk2 modulator may be targeted to selectively express in a specific population of cells, it will be understood that in addition to local administration (such as may be achieved by injection into the target tissue), the vector may be administered systemically (e.g., intravenously) in a biologically-compatible solution or pharmaceutically acceptable delivery vehicle. Vector constructs administered in this way may selectively infect the target cells or may be designed to selectively express a Pyk2 modulator in a target cells (e.g., by way of a tissue-specific promoter).

A variety of mammalian expression vectors may be used to express a Pyk2 modulator in mammalian cells. Commercially available mammalian expression vectors that may be suitable include, but are not limited to, pMClneo (Stratagene), pXTI (Stratagene), pSG5 (Stratagene), EBO-pSV2-neo (ATCC 37593) pBPV-1 (8-2) (ATCC 37110), pdBPV-MMTneo (342-12) (ATCC 37224), pRSVgpt (ATCC 37199), pRSVneo (ATCC 37198), pSV2-dhfr (ATCC 37146), pUCTag (ATCC 37460), and IZD35 (ATCC 37565). Other vectors that have been shown to be suitable expression systems in mammalian cells include the herpes simplex viral based vectors: PHSVI (Lilley et al., 2001, Curr. Gene Ther. 1:339-358; Geller et al., 1990, Proc. Natl. Acad. Sci. USA 87:8950-8954); recombinant retroviral vectors: MFG (Baragi, 2000, Curr. Opin. Mol. Ther. 2:216-220; Barranger et al., 1997, Baillieres Clin. Haematol. 10:765-778; Jaffee et al., 1993, Cancer Res. 53:2221-2226); Moloney-based retroviral vectors: LN, LNSX, LNCX, LXSN (Miller and Rosman, 1989, Biotechniques 7:980-989); vaccinia virus vectors: MVA (Sutter and Moss, 1992, Proc. Natl. Acad. Sci. USA 89:10847-10851); recombinant adenovirus vectors: pJM17 (Ishii et al., 2000, Ann. N.Y. Acad. Sci. USA 902:311-314; Ali et al., 1994, Gene Therapy 1:367-384; Berkner, 1988, Biotechniques 6:616-624); second generation adenovirus vectors: DEI/DE4 adenoviral vectors (Wang and Finer, 1996, Nature Medicine 2:714-716); and adenoassociated viral vectors: AAV/Neo (Hanazono et al., 1999, Blood 94:2263-2270; Muro-Cacho et al., 1992, J. Immunotherapy 11:231-237). Other expression vectors are disclosed, for example, by Teiger et al., 2001, Biomed. Pharmacother. 55:148-54, and by Buchschacher et al., 2001, Hum Gene Ther. 12:1013-19. Use of custom vectors based on any appropriate vector known in the art is also within the scope of the present invention.

Delivery of expression vectors to brain tissue has been described in several publications. For example, U.S. Pat. Nos. 4,866,042, 5,082,670, and 5,529,774, disclose the use of grafts or implants as one mechanism for introducing retroviral vectors bearing therapeutic gene sequences into the brain. These patents also describe an approach in which the vectors are carried across the blood brain barrier. Examples of viral vectors that can be used for the delivery of nucleic acids to the brain according to the invention include herpes simplex virus (HSV) vectors, adenovirus vectors, adenoassociated virus (AAV) vectors, and lentivirus vectors. As an example, WO 98/46273 describes gene delivery using an adenoassociated viral vector that is administered to the brain using a syringe or a catheter. WO 98/46273 also discloses intraventricular delivery in order to obtain widespread, global delivery throughout the brain.

Use of agents that enhance nucleic acid delivery to the brain is also contemplated as part of the invention. For example, intraparenchymal penetration may be enhanced by lowering brain interstitial pressure using systemic mannitol. See, e.g., PCT Patent Application No. WO 98/46273.

Alternate approaches to brain delivery of nucleic acids include injection of naked plasmid DNA as well as liposome-nucleic acid complexes. Suitable lipids and related analogs are described by U.S. Pat. Nos. 5,208,036, 5,264,618, 5,279,833 and 5,283,185. Vectors and DNA encoding an agent can also be adsorbed to or associated with particulate carriers, examples of which include polymethyl methacrylate polymers and polylactides and poly(lactide-coglycolides). See, e.g., McGee et al., 1997, J. Microencapsul. 14:197-210.

The nucleic acid vectors of the invention described above may also comprise transcription regulatory sequences such as promoters, enhancers, activators, repressors, terminators, and the like. A promoter may be such that, for example, a Pyk2 modulator is constitutively expressed or, alternatively, one in which expression is inducible. That is, the promoter may respond to a cellular signal that is always present or to a signal that is only present under certain environmental conditions. As mentioned earlier, examples of promoters that cause expression in the brain include the platelet-derived growth factor (PDGF) promoter (WO 02/26936); prion promoter (Hsiao et al., 1996, Science 274:99-102); and the neuron-specific enolase promoter (Xu et al., 2001, Gene Therapy 8:1323-1332). Examples of promoter/enhancers systems used for the expression of genes in neuronal cells include the promoter/enhancer from the immediate early (IE) human cytomegalovirus (hCMV) (WO 98/46273 and WO 99/50404), and the promoter/enhancer from the human neurofilament-light (hNF-L) gene (Vidal-Sanz et al., 1991, Eur. J. Neurosci. 3:758-763). Examples of terminators used in mammalian systems include those from the cytomegalovirus and SV40 systems, as well as the bovine growth hormone (BGH) polyadenylation sequence. See, e.g., WO 98/55616, WO 02/16594 and U.S. Pat. Nos. 6,248,555 and 6,323,030.

### Compositions

The present invention also provides compositions useful in carrying out the methods of the invention.

These compositions may contain an agent in a range from about 0.1 to about 10% of the composition. In another embodiment, these compositions may contain an agent in a range from about 0.5 to about 5% of the composition. In yet another embodiment, these compositions may contain an agent in a range from about 1 to about 3% of the composition. An agent in the context of the compositions of the invention is any molecule (nucleic acid sequence, protein, etc.) identified in this application as useful in the methods of the instant invention.

The compositions of the invention may also be used in appropriate association with other pharmaceutically active compounds. The following methods and excipients are merely exemplary and are in no way meant to be limiting.

For oral compositions, the agents can be used alone or in combination with appropriate additives to make tablets, powders, granules, or capsules, for example, with conventional additives, such as lactose, mannitol, corn starch, or potato starch; with binders, such as crystalline cellulose, cellulose derivatives, acacia, corn starch, or gelatins; with disintegrators, such as corn starch, potato starch, or sodium carboxymethylcellulose; with lubricants, such as talc or magnesium stearate; and if desired, with diluents, buffering agents, moistening agents, preservatives, and flavoring agents.

The compositions may include, depending on the composition desired, physiologically acceptable, nontoxic carriers or diluents, which are defined as vehicles commonly used to formulate compositions for animal or human administration. In general, the diluent is also pharmacologically acceptable, i.e., it does not affect the biological activity of the combination. Examples of such diluents are distilled water, physiological phosphate-buffered saline, Ringer's solution, dextrose solution, and Hank's balanced salt solution. In addition, the composition may include other carriers, adjuvants, or nontoxic, nontherapeutic, nonimmunogenic stabilizers, and the like. Also included may be carrier molecules such as proteoglycans. Specific examples of such carrier molecules include, but are not limited to, glycosaminoglycans such as heparin sulfate, hyaluronic acid, keratin-sulfate, chondroitin 4-sulfate, chondroitin 6-sulfate, heparin sulfate and dermatin sulfate, perlecan, and pento polysulfate.

The agents of the invention may be formulated into preparations for injection by dissolving, suspending, or emulsifying the agent in a physiologically acceptable diluent with a carrier. Carriers include sterile liquids, such as water, oils, with or without the addition of a surfactant, and glycols. Oils may be petroleum derivatives, or of animal, vegetable, or synthetic origin, for example, peanut oil, soybean oil, and mineral oil. Examples of glycols include propylene glycol and polyethylene glycol. The compositions may also contain conventional additives such as solubilizers, isotonic agents, suspending agents, emulsifying agents, stabilizers, and preservatives. The agents of this invention may also be used in a sustained release form, for example, a depot injection, implant preparation, or osmotic pump, which can be formulated in such a manner as to permit a sustained release of the active ingredient.

The agents can be utilized in an aerosol composition to be administered via inhalation or pulmonary delivery. The agents of the present invention may be formulated into pressurized propellants such as dichlorodifluoromethane, nitrogen, and the like.

Administration of an agent of the invention may be accomplished by any convenient means, including parenteral injection, and may be systemic or localized in delivery. The agents of this invention can be incorporated into a variety of compositions for therapeutic administration. In general, the agents of the present invention can be formulated into compositions by combination with appropriate physiologically acceptable carriers or diluents, and may be formulated into preparations in solid, semi-solid, liquid, or gaseous forms, such as tablets, capsules, powders, granules, ointments, solutions, suppositories, injections, inhalants, gels, microspheres, and aerosols. As such, administration of the agents can be achieved in various ways, including oral, buccal, rectal, parenteral, intraperitoneal, intradermal, transdermal, intratracheal, intrathecal, intranasal, gastric, intramuscular, intracranial, subdermal, etc., administration. The active agent may be systemic after administration or may be localized by the use of regional administration, intramural administration, or use of an implant that acts to retain the active component at the site of implantation.

Unit dosage forms for oral or rectal administration such as syrups, elixirs, and suspensions may be provided wherein each dosage unit, for example, teaspoonful, tablespoonful, tablet, or suppository, contains a predetermined amount of the composition containing one or more agents of the present invention. Similarly, unit dosage forms for injection or intravenous administration may comprise the agent of the present invention in a composition as a solution in sterile water, normal saline, or another pharmaceutically acceptable carrier.

Doses for nucleic acids encoding agents of the invention range from about 10 ng to about 1 g, from about 100 ng to about 100 mg, from about 1 μg to about 10 mg, or from about 30 to about 300 μg DNA per subject. Doses for infectious viral vectors vary from about 10 to about 1×10⁵ from about 100 to about 1000, from about 1000 to about 1×10⁴, or from about 1×10⁴ to about 1×10⁵, or more, virions per dose.

Those of skill in the art will readily appreciate that dose levels can vary as a function of the specific agent, the severity of the symptoms, and the susceptibility of the subject to side effects. Additionally, some of the specific agents of the invention may be more potent than others. Dosages for a given agent are readily determinable by those of skill in the art by a variety of means, for example by measuring the relative physiological potency of a given agent by methods known in the art with respect to the potency of another agent and adjusting the dosage accordingly.

Implants for sustained release compositions are well-known in the art. Implants are formulated as microspheres, slabs, etc. with biodegradable or non-biodegradable polymers. For example, polymers of lactic acid and/or glycolic acid form an erodible polymer that is well-tolerated by the host. See, e.g., Mordenti et al., 1999, Toxicol. Sci. 52:101-106; Valero et al., 1998, J. Cardiovasc. Pharmacol. 31:513-519; Blanco-Prieto et al., 1996, J. Neurochem. 67:2417-2424. The implant is, for example, placed in proximity to the site of interest (e.g., the site of formation of protein deposits associated with neurodegenerative disorders), so that the local concentration of active agent is increased at that site relative to the rest of the body.

The compositions can also be administered by infusion into the brain, and may be administered in either a continuous or non-continuous fashion. Methods, compositions, and devices suitable for delivery to the brain in a continuous (e.g., chronic) or non-continuous (e.g., single, discrete dose per administration) fashion are described in, for example, U.S. Pat. Nos. 5,711,316; 5,832,932; 5,814,014; 5,782,798; 5,752,515; 5,735,814; 5,713,923; 5,686,416; 5,624,898; 5,624,894; 5,124,146; and 4,866,042.

A typical dosage unit for administration to a subject includes, but is not limited to: a solution suitable for intravenous administration; a tablet taken from two to six times daily; or a time-release capsule or tablet taken once a day and containing a proportionally higher content of active ingredient. The time-release effect may be obtained by capsule materials that dissolve at different pH values, by capsules that release slowly by osmotic pressure, or by any other known means of controlled release.

### EXAMPLES

The following examples are included to provide those of ordinary skill in the art with a disclosure and description of how to make and use the present invention. However, these examples are not intended to limit the scope of what the inventors regard as their invention nor are they intended to represent that the experiments below are all or the only experiments performed.

### Example 1

In this example, the effect of a Pyk2 inhibitor on Aβ-induced neurotoxicity was tested in an in vitro model of AD. In addition, an inhibitor of JNK3 was tested alongside the Pyk2 inhibitor. It has been suggested that Aβ may induce neuronal apoptosis, a process which involves oxidative stress and perturbation of intracellular calcium levels. Oxidative stress is thought to activate the signal transduction pathway involving c-Jun N-terminal kinase (JNK). Activated JNK phosphorylates and activates several transcription factors including c-Jun, which in turn stimulates the transcription of several key target genes, including the death-inducer Fas ligand. The binding of Fas ligand to its receptor Fas then induces a cascade of events that ultimately lead to cell death. Inhibition of JNK3 would therefore be expected to protect neurons from Aβ-induced neurotoxicity.

A sequence encoding a dominant-negative Pyk2 was cloned into an adenoviral vector that also codes for the green fluorescent protein (GFP), which may be used as a reporter gene. The dominant-negative form of Pyk2 is a form of Pyk2 that interferes with the functioning of the normally active Pyk2. The dominant-negative form of Pyk2 comprised a lysine to alanine mutation (K457A) and the nucleic acid encoding the dominant-negative Pyk2 was inserted into the vector pADTRACK-CMV/pADEASY-1. This construct was used to generate a dominant-negative Pyk2/AD5 adenovirus. 4-week old cultures of human cortical neurons were infected with this adenovirus and the percent inhibition of Aβ-induced neurotoxicity was measured by alamar blue reading. Similarly, a sequence encoding a dominant-negative form of JNK3 was cloned into an adenoviral vector and human cortical neurons were infected. As a negative control, an adenoviral vector coding for only GFP was transduced and the percent inhibition of Aβ-induced neurotoxicity was measured.

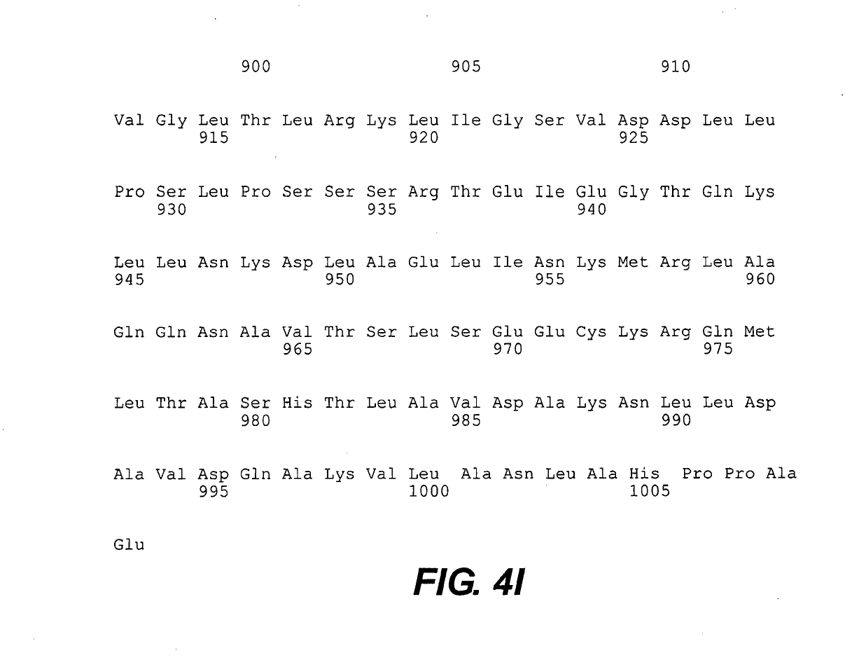

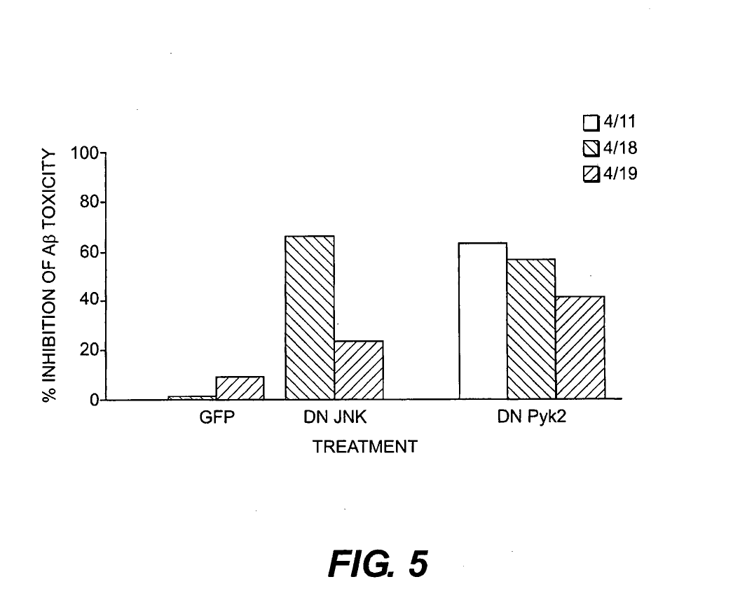

As shown in FIG. 5, the protection against Aβ-induced neurotoxicity mediated by the Pyk2 inhibitor was comparable to the protection afforded by the JNK3 inhibitor. The Pyk2 activation, however, occurred very early, i.e. within minutes, whereas JNK3 activation was observed only after many hours. This suggests that Pyk2 acts very early in the neurodegeneration pathway and that inhibitors of Pyk2 should significantly inhibit Aβ-induced neurotoxicity.

### Example 2

In this example, the effect of a Pyk2 inhibitor on stroke-induced neurodegeneration was tested in an in vitro stroke model. Rat cortical cells were infected with the adenovirus carrying the dominant-negative Pyk2 described in Example 1. The negative control used was the same as that in Example 1. Pky2 was shown to provide protection against stroke-induced neural degeneration, suggesting that Pyk2 inhibitors may be used for treating stroke.

### Example 3

In this example, the effect of a Pyk2 RNAi or antisense in stroke-induced neurodegeneration may be tested in an in vitro stroke model. A Pyk2 antisense or RNAi may be transfected into rat cortical neurons, or RNAi may be inserted into an adenoviral construct and infected into rat cortical neurons. Inhibition of Pky2 production should provide protection against stroke-induced neural degeneration, suggesting that Pyk2 RNAi or antisense may be used for treating stroke.

### Example 4

In this example, the effect of a tyrosine phosphatase which dephosphorylates Pyk2 in stroke-induced neurodegeneration may be tested in an in vitro stroke model. Rat cortical neurons may be transfected or virally infected with a vector encoding a tyrosine phosphatase. Inhibition of Pky2 tyrosine phosphorylation should provide protection against stroke-induced neural degeneration, suggesting that dephopshorylation of Pyk2 by activation of a tyrosine phosphatase may be used for treating stroke.

We claim:

1. A method for preventing cell death in a neuron comprising, administering to the neuron an effective amount of a composition comprising a Pyk2 inhibitor.

2. The method according to claim 1, wherein the cell death occurs in the presence of β-amyloid protein.

3. The method according to claim 1, wherein the cell death occurs during or after hypoxia.

4. The method according to claim 1, wherein the neuron exhibits an elevated intracellular calcium concentration.

5. The method according to claim 1, wherein the neuron is a dopaminergic neuron.

6. The method according to claim 1, wherein the Pyk2 inhibitor is a tyrphostin, quinazoline, quinaxoline, or quinoline.

7. The method according to claim 1, wherein the Pyk2 inhibitor is a dominant-negative Pyk2, an antisense nucleic acid, or an interfering RNA (RNAi).

8. The method according to claim 7, wherein the nucleic acid is an antisense oligonucleotide that decreases the expression of Pyk2.

9. The method according to claim 1, wherein the composition comprises a nucleic acid encoding a Pyk2 inhibitor.

10. The method according to claim 9, wherein the Pky2 inhibitor is a dominant-negative Pyk2, an antisense nucleic acid, or an interfering RNA (RNAi).

11. The method according to claim 9, wherein the nucleic acid encodes SEQ ID NO: 4.

12. The method according to claim 10, wherein the nucleic acid is SEQ ID NO: 3.

13. The method of claim 9, wherein the Pyk2 inhibitor is administered via a vector.

14. The method of claim 13, wherein the vector is an adenovirus vector, adenoassociated viral vector (AAV), retrovirus vector, herpes virus vector, vaccinia virus vector, or RNA virus vector.

15. The method according to claim 13, wherein the vector is a plasmid, cosmid, or yeast artificial chromosome.

16. The method according to claim 13, wherein the vector is a non-nucleic acid carrier.

17. The method according to claim 16, wherein the carrier is a lipid, lipid analog, polymethyl methacylate polymer, polyactide, or poly(lactide-co-glycolide).

18. The method according to claim 1, wherein the neuronal cell is in a patient suffering from neurodegeneration.

19. The method according to claim 18, wherein the patient suffers from Alzheimer's disease, stroke, or Parkinson's disease.
